(19) United States
(12) Patent Application Publication
(10) Pub. No.: US 2015/0175603 A1
(43) Pub. Date: Jun. 25, 2015

(54) TRICYCLIC COMPOUNDS AS KAT II INHIBITORS

(71) Applicant: PFIZER INC., NEW YORK, NY (US)

(72) Inventors: Amy Beth Dounay, Colorado Springs, CO (US); Jamison Bryce Tuttle, Marblehead, MA (US); Patrick Robert Verhoest, Newton, MA (US)

(21) Appl. No.: 14/405,103

(22) PCT Filed: Jun. 3, 2013
(86) PCT No.: PCT/IB2013/054570

§ 371 Date: Dec. 2, 2014

Related U.S. Application Data
(60) Provisional application No. 61/660,142, filed on Jun. 15, 2012.

(51) Int. Cl. C07D 471/14; C07D 471/06

(52) U.S. Cl. CPC C07D 471/14; C07D 471/06

(57) ABSTRACT

Compounds of Formula (I), wherein R¹, R², X¹, Y¹, Z¹, and Z² are as defined herein, and pharmaceutically acceptable salts thereof, are described as useful for the treatment of cognitive deficits associated with schizophrenia and other psychiatric, neurodegenerative and/or neurological disorders in mammals, including humans.

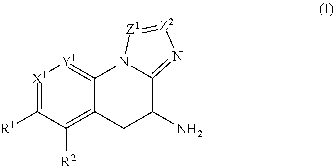

### FIELD OF THE INVENTION

The present invention generally relates to certain tricyclic compounds as inhibitors of the KAT II enzyme, which are useful for the treatment of cognitive deficits associated with schizophrenia and other psychiatric, neurodegenerative and/or neurological disorders in mammals, including humans.

### BACKGROUND OF THE INVENTION

KAT (kynurenine aminotransferase) II is a primary enzyme in the brain for catalyzing the transamination of kynurenine to KYNA (kynurenic acid) (E. Okuno et al., J. Neurochem., vol. 57, pp. 533-540, 1991). KYNA is an effective excitatory amino acid (EAA) receptor antagonist with affinity for the glycine modulatory site of the N-methyl-D-aspartate (NMDA) receptor complex (M. Kessler et al., J. Neurochem., vol. 52, pp. 1319-1328, 1989). As a naturally occurring brain metabolite, KYNA probably serves as a negative endogenous modulator of cerebral glutamatergic function (R. Schwarcz et al., Ann. N.Y. Acad. Sci., vol. 648, pp. 140-153, 1992), and activator of arylhydrocarbon receptors (B. DiNatale et al., Toxicol. Sci. vol 115, pp. 89-97, 2010).

EAA receptors and in particular NMDA receptors are known to play a central role in the function of the mammalian brain (J. C. Watkins and G. L. Collingridge, Eds., The NMDA Receptor, Oxford University Press, Oxford, 1989, p. 242). For example, NMDA receptor activation is essential for cognitive processes, such as, for example, learning and memory (Watkins and Collingridge, vide supra, pp. 137-151). Therefore, reducing KYNA synthesis by inhibition of its synthetic enzyme may enhance EAA signaling and improve cognitive processes, especially in disease states where NMDA hypofunction is anticipated. Thus, there is a need for compounds which act as KAT II inhibitors to reduce KYNA synthesis within the brain to improve cognitive dysfunction in human disease states.

### SUMMARY OF THE INVENTION

The present invention provides, in part, a compound of Formula I:

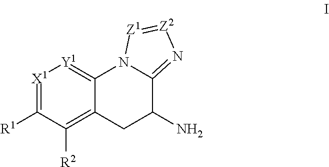

or a pharmaceutically acceptable salt thereof, wherein:

[symbol] represents a single bond or a double bond;

X¹ is CR³ or N;

Y¹ is CR⁴ or N;

Z¹ is CR⁵ or C(═O);

Z² is N, NH, or O;

R¹ is Q¹ or —O-Q¹; or —CH₂-Q¹;

R² is H, OH, —CN, halogen, optionally substituted C₁₋₄ alkyl, or optionally substituted C₁₋₄ alkoxy;

each of R³ and R⁴ is independently H, OH, —CN, halogen, optionally substituted C₁₋₄ alkyl, or optionally substituted C₁₋₄ alkoxy;

R⁵ is H, OH, —CN, C₁₋₃ alkyl optionally substituted with one or more halogen, or C₁₋₃ alkoxy optionally substituted with one or more halogen; and

Q¹ is optionally substituted phenyl or optionally substituted 5- to 10-membered heteroaryl.

This invention also provides hydrates, solvates, isomers, crystalline and non-crystalline forms, isomorphs, polymorphs, prodrugs, and metabolites of compounds of Formula I or pharmaceutically acceptable salts thereof. This invention also provides all tautomers and stereoisomers (e.g., racemates and enantiomers) of these compounds or salts. This invention also provides a pharmaceutical composition containing a compound of Formula I or a pharmaceutically acceptable salt thereof and a pharmaceutically acceptable carrier.

This invention also provides a method for treating a KAT II-mediated disorder in a mammal. Such disorders include cognitive deficits associated with schizophrenia and other psychiatric, neurodegenerative and/or neurological disorders. The method comprises administering a compound of Formula I, or a pharmaceutically acceptable salt thereof, to the mammal in an amount that is therapeutically effective to treat the disorder.

When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations to the invention, the scope of which is defined by the appended claims.

### DETAILED DESCRIPTION OF THE INVENTION

One embodiment of the present invention is a compound of Formula I as described above.

Another embodiment of the present invention is a compound of Formula I, or a pharmaceutically acceptable salt thereof, wherein R⁵ is H, OH, methyl optionally substituted with one or more halogen (e.g., methyl or CF₃), or methoxy optionally substituted with one or more halogen (e.g., methoxy or OCF₃). In a further embodiment, R⁵ is H, OH, or methyl optionally substituted with one or more halogen (e.g., methyl or CF₃). In a yet further embodiment, R⁵ is H or OH.

Another embodiment of the present invention is a compound of Formula I, or a pharmaceutically acceptable salt thereof, wherein the compound is a compound of Formula Ia, Ib, or Ic:

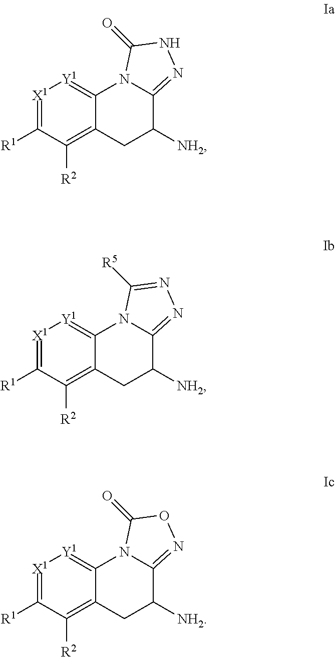

Another embodiment of the present invention is a compound of Formula I (including a compound of Formula Ia, Ib, or Ic), or a pharmaceutically acceptable salt thereof, wherein X¹ is CR³ and Y¹ is CR⁴.

Another embodiment of the present invention is a compound of Formula I (including a compound of Formula Ia, Ib, or Ic), or a pharmaceutically acceptable salt thereof, wherein X¹ is CR³ and Y¹ is N.

Another embodiment of the present invention is a compound of Formula I (including a compound of Formula Ia, Ib, or Ic), or a pharmaceutically acceptable salt thereof, wherein X¹ is N and Y¹ is CR⁴.

Another embodiment of the present invention is a compound of Formula I, or a pharmaceutically acceptable salt thereof, wherein the compound is a compound of Formula Ia-1, Ia-2, Ib-1, Ib-2, Ib-3, or Ic-1:

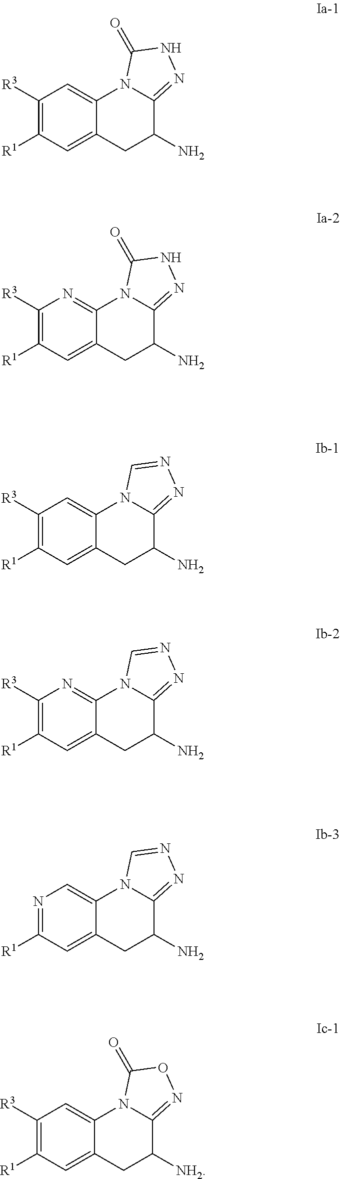

Another embodiment of the present invention is a compound of Formula I (including a compound of Formula Ia, Ib, Ic, Ia-1, Ia-2, Ib-1, Ib-2, Ib-3, or Ic-1), or a pharmaceutically acceptable salt thereof, wherein R³ is H or methyl optionally substituted with one or more halogen (e.g., methyl or CF₃). In a further embodiment, R³ is H.

Another embodiment of the present invention is a compound of Formula I (including a compound of Formula Ia, Ib, Ic, Ia-1, Ia-2, Ib-1, Ib-2, Ib-3, or Ic-1), or a pharmaceutically acceptable salt thereof, wherein R¹ is —O-Q¹; and Q¹ is optionally substituted phenyl. In a further embodiment, Q¹ is phenyl optionally substituted with one or more substituents each independently selected from the group consisting of —CN, halogen (e.g., F, Cl, or Br), C₁₋₄ alkyl optionally substituted with one or more halogen (e.g., methyl or CF₃), C₁₋₄ alkoxy optionally substituted with one or more halogen (e.g., methoxy or OCF₃), and —C(═O)—(C₁₋₄ alkyl) [e.g., —C(═O)—(CH₃)]. In a yet further embodiment, Q¹ is phenyl optionally substituted with one or more substituents each independently selected from the group consisting of —CN, F, Cl, Br, methyl, CF₃, methoxy, OCF₃, and —C(═O)—(CH₃). In a further embodiment, the para-position of the phenyl is unsubstituted or substituted with F. In a yet further embodiment, the para-position of the phenyl is unsubstituted.

Another embodiment of the present invention is a compound of Formula I (including a compound of Formula Ia, Ib, Ic, Ia-1, Ia-2, Ib-1, Ib-2, Ib-3, or Ic-1), or a pharmaceutically acceptable salt thereof, wherein R¹ is —O-Q¹; and Q¹ is phenyl optionally substituted with up to two (i.e., 0, 1, or 2) substituents each independently selected from the group consisting of —CN, halogen (e.g., F, Cl, or Br), C₁₋₄ alkyl optionally substituted with one or more halogen (e.g., methyl or CF₃), C₁₋₄ alkoxy optionally substituted with one or more halogen (e.g., methoxy or OCF₃), and —C(═O)—(C₁₋₄ alkyl) [e.g., —C(═O)—(CH₃)], and wherein each substituent on the phenyl is at one meta- or ortho-position. As used herein meta-, ortho-, or para-position of the phenyl of Q¹ is relative to the position to which the oxygen atom of the “—O-Q¹” is attached. In a further embodiment, R¹ is —O-Q¹; and Q¹ is phenyl optionally substituted at one meta-position with —CN, halogen (e.g., F, Cl, or Br), C₁₋₄ alkyl optionally substituted with one or more halogen (e.g., methyl or CF₃), C₁₋₄ alkoxy optionally substituted with one or more halogen (e.g., methoxy or OCF₃), and —C(═O)—(C₁₋₄ alkyl) [e.g., —C(═O)—(CH₃)].

Another embodiment of the present invention is a compound of Formula I (including a compound of Formula Ia, Ib, Ic, Ia-1, Ia-2, Ib-1, Ib-2, Ib-3, or Ic-1), or a pharmaceutically acceptable salt thereof, wherein R¹ is Q¹ or —CH₂-Q¹. In a further embodiment, R¹ is optionally phenyl or benzyl, wherein the phenyl moiety of the benzyl is an optionally substituted phenyl. In a yet further embodiment, R¹ is phenyl or benzyl, wherein the phenyl or the phenyl moiety of the benzyl is optionally substituted with one or more substituents each independently selected from the group consisting of —CN, halogen (e.g., F, Cl, or Br), C₁₋₄ alkyl optionally substituted with one or more halogen (e.g., methyl or CF₃), C₁₋₄ alkoxy optionally substituted with one or more halogen (e.g., methoxy or OCF₃), and —C(═O)—(C₁₋₄ alkyl) [e.g., —C(═O)—(CH₃)].

Another embodiment of the present invention is a compound of Formula I (including a compound of Formula Ia, Ib, Ic, Ia-1, Ia-2, Ib-1, Ib-2, Ib-3, or Ic-1), or a pharmaceutically acceptable salt thereof, wherein R¹ is phenyl optionally substituted with up to two substituents each independently selected from the group consisting of —CN, halogen (e.g., F, Cl, or Br), C₁₋₄ alkyl optionally substituted with one or more halogen (e.g., methyl or CF₃), C₁₋₄ alkoxy optionally substituted with one or more halogen (e.g., methoxy or OCF₃), and —C(═O)—(C₁₋₄ alkyl) [e.g., —C(═O)—(CH₃)]. In a further embodiment, the para-position of the phenyl is unsubstituted or substituted with F. In a yet further embodiment, the para-position of the phenyl is unsubstituted. In a still further embodiment, each substituent on the phenyl of R¹ is at one meta- or ortho-position. As used herein, para-, meta- or ortho-position of the phenyl of R¹ is relative to the position of the phenyl to which the tricyclic ring of Formula I is attached. In a further embodiment, R¹ is phenyl optionally substituted at one meta-position with halogen (e.g., F, Cl, or Br), C₁₋₄ alkyl optionally substituted with one or more halogen (e.g., methyl or CF₃), C₁₋₄ alkoxy optionally substituted with one or more halogen (e.g., methoxy or OCF₃), and —C(═)—(C₁₋₄ alkyl) [e.g., —C(═O)—(CH₃)].

Another embodiment of the present invention is a compound of Formula I (including a compound of Formula Ia, Ib, Ic, Ia-1, Ia-2, Ib-1, Ib-2, Ib-3, or Ic-1), or a pharmaceutically acceptable salt thereof, wherein R¹ is optionally substituted 5- to 10-membered heteroaryl. In a further embodiment, R¹ is 5- to 10-membered heteroaryl optionally substituted with one or more substituents each independently selected from the group consisting of halogen (e.g., F, Cl, or Br), C₁₋₄ alkyl optionally substituted with one or more halogen (e.g., methyl or CF₃), C₁₋₄ alkoxy optionally substituted with one or more halogen (e.g., methoxy or OCF₃), and —C(═O)—(C₁₋₄ alkyl) [e.g., —C(═O)—(CH₃)]. In a further embodiment, R¹ is pyridinyl, pyrazolyl, indolyl, or indazolyl, each optionally substituted with one or more substituents each independently selected from the group consisting of halogen (e.g., F, Cl, or Br), C₁₋₄ alkyl optionally substituted with one or more halogen (e.g., methyl or CF₃), C₁₋₄ alkoxy optionally substituted with one or more halogen (e.g., methoxy or OCF₃), and —C(═O)—(C₁₋₄ alkyl) [e.g., —C(═O)—(CH₃)].

Another embodiment of the present invention is a compound of Formula I (including a compound of Formula Ia, Ib, Ic, Ia-1, Ia-2, Ib-1, Ib-2, Ib-3, or Ic-1), or a pharmaceutically acceptable salt thereof, wherein R¹ is pyridin-3-yl optionally substituted with up to two substituents each independently selected from the group consisting of halogen (e.g., F, Cl, or Br), C₁₋₄ alkyl optionally substituted with one or more halogen (e.g., methyl or CF₃), C₁₋₄ alkoxy optionally substituted with one or more halogen (e.g., methoxy or OCF₃), and —C(═O)—(C₁₋₄ alkyl) [e.g., —C(═O)—(CH₃)]. In a further embodiment, each substituent on the pyridin-3-yl is at the 2-, 5-, or 6-position. In another further embodiment, the 4-position of the pyridin-3-yl is unsubstituted or substituted with F. In a still further embodiment, R¹ is pyridin-3-yl substituted at the 2-position with halogen (e.g., methyl or CF₃), C₁₋₄ alkoxy optionally substituted with one or more halogen (e.g., methoxy or OCF₃), or —C(═O)—(C₁₋₄ alkyl) [e.g., —C(═O)—(CH₃)].

In some embodiments of the compound of Formula I (including a compound of Formula Ia, Ib, Ic, Ia-1, Ia-2, Ib-1, Ib-2, Ib-3, or Ic-1) or a pharmaceutically acceptable salt thereof, the carbon atom to which the NH₂ is attached has the R configuration.

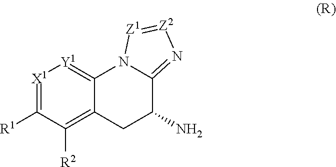

In some other embodiments, the carbon atom to which the NH₂ is attached has the S configuration.

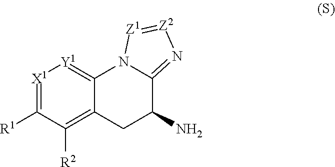

In one embodiment, the invention also provides each compound, individually, described in Examples 1 to 48 discussed herein (including all racemates, enantiomers/stereoisomers, free bases, and pharmaceutically acceptable salts thereof).

The present invention comprises the tautomeric forms of compounds of Formula I (including a compound of Formula Ia, Ib, Ic, Ia-1, Ia-2, Ib-1, Ib-2, Ib-3, or Ic-1). Where structural isomers are interconvertible via a low energy barrier, tautomeric isomerism (‘tautomerism’) can occur. This can take the form of proton tautomerism in compounds of Formula I containing, for example, an imino, keto, or oxime group, or so-called valence tautomerism in compounds which contain an aromatic moiety. It follows that a single compound may exhibit more than one type of isomerism. The various ratios of the tautomers in solid and liquid form is dependent on the various substituents on the molecule as well as the particular crystallization technique used to isolate a compound. It is understood that the compounds of the present invention include all tautomeric forms even where only one of the tautomeric forms is shown. For example, the present invention includes compounds of both Formula Ia and Ia′

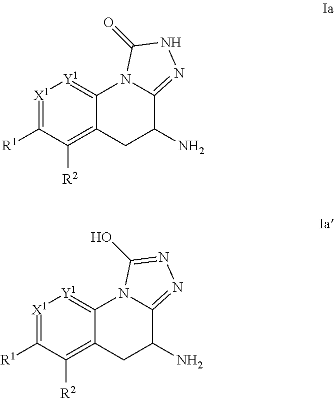

even where only Formula Ia is shown.

Another embodiment of the present invention is a method for the treatment in a mammal of a KAT II-mediated condition or disorder selected from the group consisting of acute neurological and psychiatric disorders; stroke; cerebral ischemia; spinal cord trauma; cognitive impairment, including mild cognitive impairment; head trauma; perinatal hypoxia; cardiac arrest; hypoglycemic neuronal damage; dementia; Alzheimer's disease; Huntington's Chorea; amyotrophic lateral sclerosis; ocular damage; retinopathy; cognitive disorders; idiopathic and drug-induced Parkinson's disease; muscular spasms and disorders associated with muscular spasticity including tremors; epilepsy; convulsions; migraine; urinary incontinence; substance tolerance; substance withdrawal; psychosis; schizophrenia; negative symptoms associated with schizophrenia; autism, including autism spectrum disorders; bipolar disorder; depression, including but not limited to Major Depressive Disorder and treatment-resistant depression; cognitive impairment associated with depression; cognitive impairment associated with cancer therapy; anxiety; mood disorders; inflammatory disorders; sepsis; cirrhosis; cancer and/or tumors associated with immune response escape; trigeminal neuralgia; hearing loss; tinnitus; macular degeneration of the eye; emesis; brain edema; pain; tardive dyskinesia; sleep disorders; attention deficit/hyperactivity disorder; attention deficit disorder; disorders that comprise as a symptom of deficiency in attention and/or cognition; and conduct disorder; which method comprises administering to the mammal a compound of Formula I (including a compound of Formula Ia, Ib, Ic, Ia-1, Ia-2, Ib-1, Ib-2, Ib-3, or Ic-1) or a pharmaceutically acceptable salt thereof. In another embodiment, the invention provides use of one or more compounds of the invention or salts thereof for the treatment of the conditions/disorders recited herein. In another embodiment, the invention provides use of one or more compounds of the invention or salts thereof for the preparation of a medicament for the treatment of the conditions/disorders recited herein.

Another embodiment of the present invention is a method for the treatment in a mammal of a condition/disorder selected from the group consisting of dementia; cognitive deficit symptoms of Alzheimer's disease; attention deficit symptoms of Alzheimer's disease; multi-infarct dementia, alcoholic dementia or other drug-related dementia, dementia associated with intracranial tumors or cerebral trauma, dementia associated with Huntington's disease or Parkinson's disease, or AIDS-related dementia; delirium; amnestic disorder; post-traumatic stress disorder; mental retardation; a learning disorder (e.g., reading disorder, mathematics disorder, or a disorder of written expression); attention-deficit/hyperactivity disorder; age-related cognitive decline; cognitive deficits associated with psychoses; or cognitive deficits associated with schizophrenia, which method comprises administering to the mammal a compound of Formula I (including a compound of Formula Ia, Ib, Ic, Ia-1, Ia-2, Ib-1, Ib-2, Ib-3, or Ic-1) or a pharmaceutically acceptable salt thereof. In another embodiment, the invention provides use of one or more compounds of the invention or salts thereof for the treatment of the conditions/disorders recited herein. In another embodiment, the invention provides the use of one or more compounds of the invention or salts thereof for the preparation of a medicament for the treatment of the conditions/disorders recited herein.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting/retarding the disease; for example, inhibiting/retarding progression of a disease, condition or disorder in an individual who is experiencing or displaying the pathology or symptomatology of the disease, condition or disorder; and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or completely eliminating/curing the disease. As used herein, treating a disease further includes treating one or more symptoms associated with the disease.

Prodrugs of the compounds of Formula I can, when administered into or onto the body, be converted into compounds of Formula I or pharmaceutically acceptable salts thereof having the desired activity.

### Abbreviations and Definitions

As used throughout this specification and the appended claims, the following terms have the following meanings:

The term “C₁₋₄ alkyl” as used herein, means a straight or branched chain hydrocarbon containing from 1 to 4 carbon atoms. Examples of C₁₋₄ alkyl include methyl, ethyl, n-propyl, iso-propyl, n-butyl, sec-butyl, iso-butyl, tert-butyl.

The term “C₁₋₃ alkyl” as used herein, means a straight or branched chain hydrocarbon containing from 1 to 3 carbon atoms. Examples of C₁₋₃ alkyl include methyl, ethyl, n-propyl,and iso-propyl.

The term “C₁₋₄ alkoxy” as used herein, means an —O—C₁₋₄ alkyl group, wherein the C₁₋₄ alkyl is as defined herein. Examples of C₁₋₄ alkoxy include methoxy, ethoxy, propoxy, 2-propoxy, butoxy, and tert-butoxy.

The term “C₁₋₃ alkoxy” as used herein, means an —O—C₁₋₃ alkyl group, wherein the C₁₋₃ alkyl is as defined herein. Examples of C₁₋₃ alkoxy include methoxy, ethoxy, propoxy, and 2-propoxy.

The term “benzyl” as used herein, means a —CH₂-phenyl group.

The term “halo” or “halogen” as used herein, means —F, —Cl, —Br, or —I.

As used herein, the term “heteroaryl” refers to monocyclic or fused-ring polycyclic aromatic heterocyclic groups with one or more heteroatom ring members (ring-forming atoms) each independently selected from O, S and N in at least one ring. The term “5- to 10-membered heteroaryl” as used herein, means a 5- or 6-membered monocyclic heteroaryl or a 8- to 10-membered bicyclic heteroaryl. The heteroaryl group can also contain one to three oxo groups. In some embodiments, a 5 membered heteroaryl comprises two double bonds and one, two, three or four nitrogen atoms and/or optionally one oxygen or sulfur atom. In some embodiments, a 6 membered heteroaryl comprises three double bonds and one, two, three, or four nitrogen atoms. The 5 or 6 membered heteroaryl is connected to the parent molecular moiety through any carbon atom or any nitrogen atom contained within the heteroaryl. Examples of monocyclic heteroaryl include furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. A bicyclic heteroaryl comprises a monocyclic heteroaryl fused to a phenyl or a monocyclic heteroaryl fused to a monocyclic heteroaryl. The bicyclic heteroaryl is connected to the parent molecular moiety through any carbon atom or any nitrogen atom contained within the bicyclic heteroaryl. Examples of bicyclic heteroaryl include benzimidazolyl, benzofuranyl, benzothienyl, benzoxadiazolyl, benzoxazolyl, benzothiazolyl, furopyridinyl, indolyl, indazolyl, isoquinolinyl, naphthyridinyl, phthalazinyl, pyrrolopyridinyl, quinazolinyl, quinolinyl, quinoxalinyl, and thienopyridinyl.

As used herein, the term “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group (up to that every hydrogen atom on the designated atom or moiety is replaced with a selection from the indicated substituent group), provided that the normal valency of the designated atom or moiety is not exceeded, and that the substitution results in a stable compound. For example, if a methyl group (i.e., CH₃) is optionally substituted, then up to 3 hydrogen atoms on the carbon atom can be replaced with substituent groups. If an atom or moiety is described as being optionally substituted with one or more non-hydrogen substituents, then it can be substituted by up the maximum number of substitutable positions on the atom or moiety.

If an atom or moiety is described as being optionally substituted with up to a particular number of non-hydrogen substituents, then that atom or moiety may be either (1) not substituted; or (2) substituted by up to that particular number of non-hydrogen substituents or by up to the maximum number of substitutable positions on the atom or moiety, whichever is less. Thus, for example, if a moiety is described as heteroaryl optionally substituted with up to 2 non-hydrogen substituents, then any heteroaryl with less than 2 substitutable positions would be optionally substituted by up to only as many non-hydrogen substituents as the heteroaryl has substitutable positions. To illustrate, tetrazolyl (which has only one substitutable position) would be optionally substituted with up to one non-hydrogen substituent.

If substituents are described as being “independently selected” from a group, each substituent is selected independent of the other. Each substituent therefore may be identical to or different from the other substituent(s).

The term “optionally substituted C₁₋₄ alkyl” as used herein, means a C₁₋₄ alkyl group optionally substituted with one or more substituents each independently selected from the group consisting OH, —CN, NO₂, halogen, and C₁₋₄ alkoxy optionally substituted one or more halogen.

The term “optionally substituted C₁₋₄ alkoxy” as used herein, means a C₁₋₄ alkoxy group optionally substituted with one or more substituents each independently selected from the group consisting OH, —CN, NO₂, halogen, and C₁₋₄ alkoxy optionally substituted one or more halogen.

As used herein, the term “optionally substituted 5- to 10-membered heteroaryl heteroaryl” refers to a 5- to 10-membered heteroaryl group optionally substituted with one or more (e.g., 1, 2, 3, 4, or 5) groups each independently selected from the group consisting of OH, —CN, NO₂, halogen, optionally substituted C₁₋₄ alkyl, optionally substituted C₁₋₄ alkoxy, —NH₂, —NH(C₁₋₄ alkyl), —N(C₁₋₄ alkyl)₂, and —C(═O)—(C₁₋₄ alkyl). Heteroaryl groups of the invention that are optionally substituted may be as tautomers. The present invention encompasses all tautomers including non-aromatic tautomers.

As used herein, the term “optionally substituted phenyl” refers to a phenyl group optionally substituted with one or more (e.g., 1, 2, 3, 4, or 5) groups each independently selected from the group consisting of OH, —CN, NO₂, halogen, optionally substituted C₁₋₄ alkyl, optionally substituted C₁₋₄ alkoxy, —NH₂, —NH(C₁₋₄ alkyl), —N(C₁₋₄ alkyl)₂, and —C(═O)—(C₁₋₄ alkyl).

As used herein the term “Formula I” may be referred to as “a compound of the invention” or as “compounds of the invention.” Such terms are also defined to include all forms of the compound of Formula I, including hydrates, solvates, stereoisomers (e.g., diastereomeric, enantiomeric, and epimeric forms as well as racemates and mixtures thereof), tautomers, crystalline and non-crystalline forms, isomorphs, polymorphs, prodrugs and metabolites thereof.

### Isomers

When an asymmetric center is present in a compound of Formula I, hereinafter referred to as the compound of the invention, the compound may exist in the form of optical isomers (e.g., enantiomers). In one embodiment, the present invention comprises enantiomers and mixtures, including racemic mixtures of the compounds of Formula I. In another embodiment, for compounds of Formula I that contain more than one asymmetric center, the present invention comprises diastereomeric forms (individual diastereomers and/or mixtures thereof) of compounds. When a compound of Formula I contains an alkenyl group, geometric isomers (e.g., cis, trans, E, or Z forms) may arise.

### Salts

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above. Some examples of salts of the present invention include trifluoroacetate (CF₃CO₂H), tosylate (CH₃C₆H₄SO₂OH), sulfate (H₂SO₄), and hydrochloride (HCl).

The invention also relates to base addition salts of the compounds of the invention. The chemical bases that may be used as reagents to prepare these pharmaceutically acceptable base salts are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Suitable base salts are formed from bases which form non-toxic salts. Non-limiting examples of suitable base salts include the aluminum, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002). Methods for making pharmaceutically acceptable salts of compounds of the invention are known to one of skill in the art.

### Isotopes

The present invention also includes isotopically labeled compounds, which are identical to those recited in Formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorus, sulfur, fluorine and chlorine, such as ²H, ³H, ¹³C, ¹¹C, ¹⁴C, ¹⁵N, ¹⁸O, ¹⁷O, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula I of this invention and prodrugs thereof can generally be prepared by carrying out the procedures disclosed in the Schemes and/or in the Examples and Preparations below, by substituting a readily available isotopically labeled reagent for a non-isotopically labeled reagent.

The invention also relates to prodrugs of the compounds of Formula I. Thus certain derivatives of compounds of Formula I which may have little or no pharmacological activity themselves can, when administered into or onto the body, be converted into compounds of Formula I having the desired activity, for example, by hydrolytic cleavage. Such derivatives are referred to as “prodrugs”. Further information on the use of prodrugs may be found in Prodrugs as Novel Delivery Systems, Vol. 14, ACS Symposium Series (T. Higuchi and V. Stella) and Bioreversible Carriers in Drug Design, Pergamon Press, 1987 (Ed., E. B. Roche, American Pharmaceutical Association).

### Prodrugs and Metabolites

The invention also relates to prodrugs of the compounds of the invention. Thus certain derivatives of compounds of the invention which may have little or no pharmacological activity themselves can, when administered into or onto the body, be converted into compounds of the invention having the desired activity, for example, by hydrolytic cleavage. Such derivatives are referred to as “prodrugs”. Further information on the use of prodrugs may be found in Prodrugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Some non-limiting examples of prodrugs in accordance with the invention include:
(i) where the compound of Formula I contains a carboxylic acid functionality which is functionalized into a suitably metabolically labile group (esters, carbamates, etc.) on the compound of Formula I;
(ii) where the compound of Formula I contains an alcohol functionality which is functionalized into a suitably metabolically labile group (esters, carbonates, carbamates, acetals, ketals, etc.) on the compound of Formula I; and
(iii) where the compound of Formula I contains a primary or secondary amino functionality, or an amide which is functionalized into a suitably metabolically labile group, e.g., a hydrolyzable group (amides, carbamates, ureas, etc.) on the compound of Formula I.

Further examples of replacement groups in accordance with the foregoing examples and examples of other prodrug types may be found in the aforementioned references.

Moreover, certain compounds of Formula I may themselves act as prodrugs of other compounds of Formula I.

Also included within the scope of the invention are metabolites of compounds of Formula I, that is, compounds formed in vivo upon administration of the drug.

### Administration and Dosing

Typically, a compound of the invention or a pharmaceutically acceptable salt thereof is administered in an amount effective to treat a condition as described herein. The compounds of the invention or salts thereof are administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts.

The compounds of the invention or salts thereof may be administered orally. Oral administration may involve swallowing, so that the compound enters the gastrointestinal tract, or buccal or sublingual administration may be employed by which the compound enters the bloodstream directly from the mouth.

In another embodiment, the compounds of the invention may also be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In another embodiment, the compounds of the invention may also be administered topically to the skin or mucosa, that is, dermally or transdermally. In another embodiment, the compounds of the invention can also be administered intranasally or by inhalation. In another embodiment, the compounds of the invention may be administered rectally or vaginally. In another embodiment, the compounds of the invention may also be administered directly to the eye or ear.

The dosage regimen for the compounds and/or compositions containing the compounds or salts thereof is based on a variety of factors, including the type, age, weight, sex and medical condition of the patient; the severity of the condition; the route of administration; and the activity of the particular compound employed. Thus the dosage regimen may vary widely. Dosage levels of the order from about 0.01 mg to about 100 mg per kilogram of body weight per day are useful in the treatment of the above-indicated conditions. In one embodiment, the total daily dose of a compound of the invention (administered in single or divided doses) is typically from about 0.01 to about 100 mg/kg. In another embodiment, total daily dose of the compound of the invention is from about 0.1 to about 50 mg/kg, and in another embodiment, from about 0.5 to about 30 mg/kg (i.e., mg compound of the invention per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound will be repeated a plurality of times in a day (typically no greater than 4 times). Multiple doses per day typically may be used to increase the total daily dose, if desired.

For oral administration, the compositions may be provided in the form of tablets containing for example 0.01, 0.05, 0.1, 0.5, 1.0, 2.5, 5.0, 10.0, 15.0, 25.0, 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

For the treatment of the conditions referred to herein, the compound of the invention can be administered as compound per se. Alternatively, pharmaceutically acceptable salts are suitable for medical applications because of their greater aqueous solubility relative to the parent compound.

### Pharmaceutical Compositions

In another embodiment, the present invention provides pharmaceutical compositions. Such a pharmaceutical composition comprises a compound of the invention or a pharmaceutically acceptable salt thereof and a pharmaceutically acceptable carrier. The carrier can be a solid, a liquid, or both, and may be formulated with the compound as a unit-dose composition, for example, a tablet, which can contain from 0.05% to 95% by weight of the active compounds. A compound of the invention may be coupled with suitable polymers as targetable drug carriers. Other pharmacologically active substances can also be present.

The pharmaceutically acceptable carrier may comprise any conventional pharmaceutical carrier or excipient. Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid, may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration, the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulation, solution or suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages. One of ordinary skill in the art would appreciate that the composition may be formulated in sub-therapeutic dosage such that multiple doses are envisioned.

The compounds of the present invention may be administered by any suitable route, for example in the form of a pharmaceutical composition adapted to such a route, and in a dose effective for the treatment intended. The active compounds and compositions, for example, may be administered orally, rectally, parenterally, or topically.

Oral administration of a solid dose form may be, for example, presented in discrete units, such as hard or soft capsules, pills, cachets, lozenges, or tablets, each containing a predetermined amount of at least one compound of the present invention. In another embodiment, the oral administration may be in a powder or granule form. In another embodiment, the oral dose form is sub-lingual, such as, for example, a lozenge. In such solid dosage forms, the compounds of Formula I are ordinarily combined with one or more adjuvants. Such capsules or tablets may contain a controlled-release formulation. In the case of capsules, tablets, and pills, the dosage forms also may comprise buffering agents or may be prepared with enteric coatings.

In another embodiment, oral administration may be in a liquid dose form. Liquid dosage forms for oral administration include, for example, pharmaceutically acceptable emulsions, solutions, suspensions, syrups, and elixirs containing inert diluents commonly used in the art (i.e., water). Such compositions also may comprise adjuvants, such as wetting, emulsifying, suspending, flavoring (e.g., sweetening), and/or perfuming agents.

In another embodiment, the present invention comprises a parenteral dose form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting agents, and/or suspending agents.

In another embodiment, the present invention comprises a topical dose form. “Topical administration” includes, for example, transdermal administration, such as via transdermal patches or iontophoresis devices, via intraocular administration, via topical ocular administration, or via intranasal or inhalation administration. Compositions for topical administration also include, for example, topical gels, sprays, ointments, and creams. A topical formulation may include a compound which enhances absorption or penetration of the active ingredient through the skin or other affected areas. When the compounds of this invention are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, B. C. Finnin and T. M. Morgan, J. Pharm. Sci., vol. 88, pp. 955-958, 1999.

Formulations suitable for topical administration to the eye include, for example, eye drops wherein the compound of this invention is dissolved or suspended in a suitable carrier. A typical formulation suitable for ocular or aural administration may be in the form of drops of a micronized suspension or solution in isotonic, pH-adjusted, sterile saline. Other formulations suitable for ocular and aural administration include ointments, biodegradable (i.e., absorbable gel sponges, collagen) and non-biodegradable (i.e., silicone) implants, wafers, lenses and particulate or vesicular systems, such as niosomes or liposomes. A polymer such as crossed-linked polyacrylic acid, polyvinyl alcohol, hyaluronic acid, a cellulosic polymer, for example, hydroxypropylmethylcellulose, hydroxyethylcellulose, or methylcellulose, or a heteropolysaccharide polymer, for example, gelan gum, may be incorporated together with a preservative, such as benzalkonium chloride. Such formulations may also be delivered by iontophoresis.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant. Formulations suitable for intranasal administration are typically administered in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (for example an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

In another embodiment, the present invention comprises a rectal dose form. Such rectal dose form may be in the form of, for example, a suppository. Cocoa butter is a traditional suppository base, but various alternatives may be used as appropriate.

Other carrier materials and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3rd Ed.), American Pharmaceutical Association, Washington, 1999.

### Co-Administration

The compounds of the present invention can be used, alone or in combination with other therapeutic agents, in the treatment of various conditions or disease states. The compound(s) of the present invention and other therapeutic agent(s) may be may be administered simultaneously (either in the same dosage form or in separate dosage forms) or sequentially. An exemplary therapeutic agent may be, for example, a metabotropic glutamate receptor agonist.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

In one embodiment, the compounds of this invention are administered as adjunctive therapy with known anti-psychotics such as Ziprasidone (Geodon), Clozapine, Molindone, Loxapine, Pimozide, Risperidone, Olanzapine, Remoxipride, Sertindole, Amisulpride, Quetiapine, Prochlorperazine, Fluphenazine, Trifluoroperazine, Thioridazine, Haloperidol, Chlorpromazine, Flupentixol and Pipotiazine.

In another embodiment, the compounds of the present invention may also be used in combination with CNS agents such as antidepressants (such as sertraline), anti-Parkinsonian drugs (such as deprenyl, L-dopa, Requip, Mirapex, MAOB inhibitors such as selegiline and rasagiline, COMT inhibitors such as Tasmar, A-2 inhibitors, dopamine reuptake inhibitors, NMDA antagonists, nicotine agonists, dopamine agonists and inhibitors of neuronal nitric oxide synthase), anti-Alzheimer's drugs such as donepezil, tacrine, alpha2delta inhibitors, COX-2 inhibitors, gaba pentenoids, propentofylline or metrifonate, and antipyschotics such as PDE10 inhibitors, 5HT2C agonists, alpha 7 nicotinic receptor agonists, CB1 antagonists and compounds having activity antagonizing dopamine D2 receptors.

### Kits

The present invention further comprises kits that are suitable for use in performing the methods of treatment described above. In one embodiment, the kit contains a first dosage form comprising one or more of the compounds of the present invention and a container for the dosage, in quantities sufficient to carry out the methods of the present invention.

In another embodiment, the kit of the present invention comprises one or more compounds of the invention.

### Preparations

In another embodiment, the invention relates to the novel intermediates useful for preparing the compounds of the invention.

The compounds of Formula I or salts thereof may be prepared by the methods described below, together with synthetic methods known in the art of organic chemistry, or modifications and transformations that are familiar to those of ordinary skill in the art. The starting materials used herein are commercially available or may be prepared by routine methods known in the art [such as those methods disclosed in standard reference books such as the Compendium of Organic Synthetic Methods, Vol. I-XII (published by Wiley-Interscience)]. Exemplary methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991; and T. W. Greene and P. G. M. Wuts, Protective Groups in Organic Chemistry, John Wiley & Sons, 1999, which are hereby incorporated by reference.

Compounds of Formula I, and/or their pharmaceutically acceptable salts, can be prepared according to the reaction Schemes discussed herein below. Unless otherwise indicated, the substituents in the Schemes are defined as above. Isolation and purification of the products is accomplished by standard procedures, which are known to a chemist of ordinary skill.

It will be understood by one skilled in the art that the various symbols, superscripts and subscripts used in the schemes, methods and examples are used for convenience of representation and/or to reflect the order in which they are introduced in the schemes, and are not intended to necessarily correspond to the symbols, superscripts or subscripts in the appended claims. The schemes are representative of methods useful in synthesizing the compounds of the present invention. They are not to constrain the scope of the invention in any way.

Scheme 1 refers to preparation of compounds of Formula I or salts thereof. Referring to Scheme 1, compounds of Formula 1-1 or 1-2 (wherein Pg is a suitable protecting group, such as Boc or Cbz) are commercially available or can be made by methods described herein or other methods well known to those skilled in the art. Compounds of Formula 1-2 are commercially available as the individual enantiomers. A compound of Formula 1-3 can be prepared by coupling a compound of Formula 1-1 with a compound of Formula 1-2, for example, by initial conversion of 1-2 to a zincate intermediate by reaction with zinc metal that has been activated (e.g., with iodine) in a suitable solvent, such as N,N-dimethylformamide, and subsequent treatment with a compound of Formula 1-1 and a suitable metal catalyst [such as a palladium catalyst, e.g., Pd(OAc)₂] and ligand [such as X-Phos] [J. B. Tuttle et al., Tetrahedron Lett. 2011, 52, 5211-5213]. A compound of Formula 1-3 can be subsequently converted to a compound of Formula 1-4 by reaction with appropriate reagents, such as P₂S₅ with sodium carbonate, in an appropriate solvent, such as tetrahydrofuran (THF). Alternatively, the same transformation can be achieved using Lawesson's reagent [2,4-bis(4-methoxyphenyl)-1,3,2,4-dithiadiphosphetane-2,4-dithione] in an appropriate solvent, such as toluene. A compound of Formula 1-4 can be converted to a compound of Formula 1-5 using an appropriate reagent, such as methyl iodide in the presence of base (e.g., potassium carbonate) in an appropriate solvent, such as THF. A compound of Formula 1-5 can be converted to a compound of Formula 1-7 using methods described herein or by other methods known to those skilled in the art. For example, a compound of Formula 1-5 can be treated with a hydrazinecarboxylate reagent [NH₂NHC(═O)R¹⁰¹] in which R¹⁰¹ is alkoxy, such as ethyl hydrazinecarboxylate, in an appropriate solvent, such as methanol or ethanol, to form an intermediate product of Formula 1-6, wherein Z¹ is C(═O) and Z² is NH, which is transferred to another solvent, such as DMF or acetonitrile, and heated under conventional or microwave heating conditions to provide a compound of Formula 1-7 (wherein Z¹ is C(═O) and Z² is NH). Alternatively, a compound of Formula 1-4 can be converted directly to a compound of Formula 1-7 by use of appropriate reaction conditions, as described herein or by other methods known to those skilled in the art. For example, conversion of a compound of Formula 1-4 to a compound of Formula 1-7 can be accomplished by heating with a hydrazine reagent, such as ethyl hydrazinecarboxylate [to form compound of Formula 1-7 wherein Z¹ is C(═O) and Z² is NH] or formic hydrazide (e.g., to form to form compound of Formula 1-7 wherein Z¹ is CR⁵, Z² is N, and R⁵ is H), in the presence of additional reagents to promote the reaction, such as magnesium sulfate and/or acetic acid, in an appropriate solvent, such as cyclohexanol. For compounds of Formula 1-7 wherein Z¹ is CR⁵ and R⁵ is other than H, the R⁵ group may be incorporated using a modification based on known methodology [K. Sharma and P. S. Fernandes, Indian J. Heterocyclic Chem. 2005, 15, 161-168.] Removal of the protecting group from compounds of Formula 1-7 under conditions well-known to those skilled in the art affords compounds of Formula I.

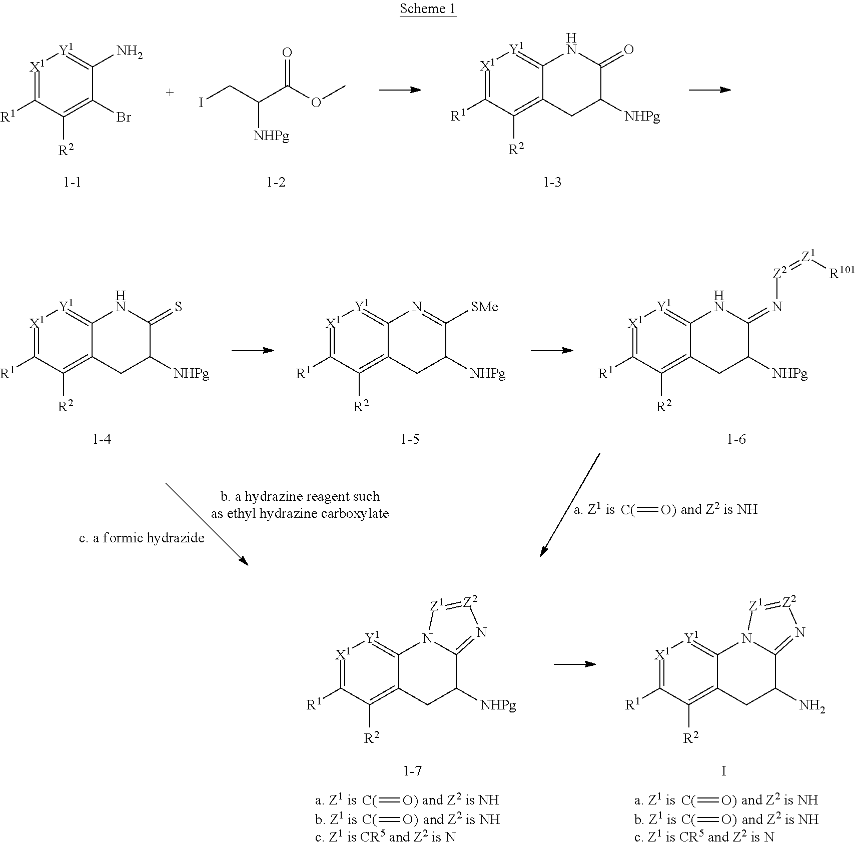

Scheme 2 refers to an alternative method for preparing compounds of Formula 1-3, which can be used to prepare a compound of Formula I (or an intermediate such as a compound of Formula 1-4) using methods such as those shown in Scheme 1. Compounds of Formula 2-1 and 2-2 are commercially available or can be made by methods described herein or other methods well known to those skilled in the art. Referring to Scheme 2, a nitroaromatic or nitroheteroaromatic starting reagent of Formula 2-1 can be converted to a compound of Formula 2-3 using methods analogous to those described in Scheme 1 for the conversion of a compound of Formula 1-1 to a compound of Formula 1-3. A compound of Formula 2-3 can be converted to a compound of Formula 1-3 using appropriate reduction methods, such as the methods described herein (e.g., using Zn and NH₄Cl) or other methods well known to those skilled in the art.

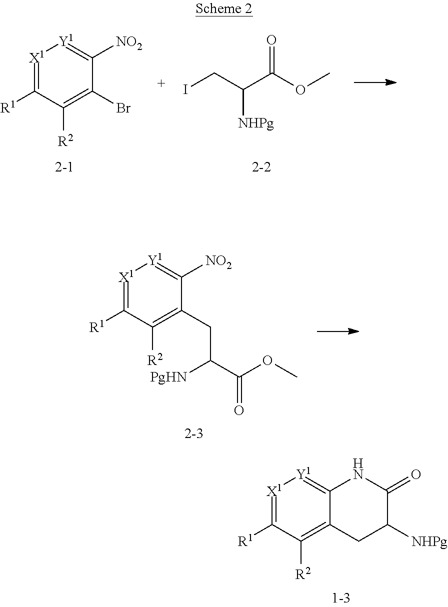

Scheme 3 refers to a preparation of compounds of Formula I wherein Z¹ is C(═O) and Z² is O. Compounds of Formula 1-4 can be prepared as described in Schemes 1 or 2. A compound of Formula 3-1 can be prepared by treating a thioamide of Formula 1-4 with hydroxylamine hydrochloride and an appropriate base, such as sodium bicarbonate, in an appropriate solvent, such as methanol. A compound of Formula 1-7 (wherein Z¹ is C(═O) and Z² is O) can be prepared by treating a compound of Formula 3-1 with an appropriate reagent, such as 1,1′-carbonyldiimidazole, in a suitable solvent, such as dichloromethane, or by using other reagents well known to those skilled in the art. Removal of the protecting group from compounds of Formula 1-7 under conditions well-known to those skilled in the art affords compounds of Formula I (wherein Z¹ is C(═O) and Z² is O).

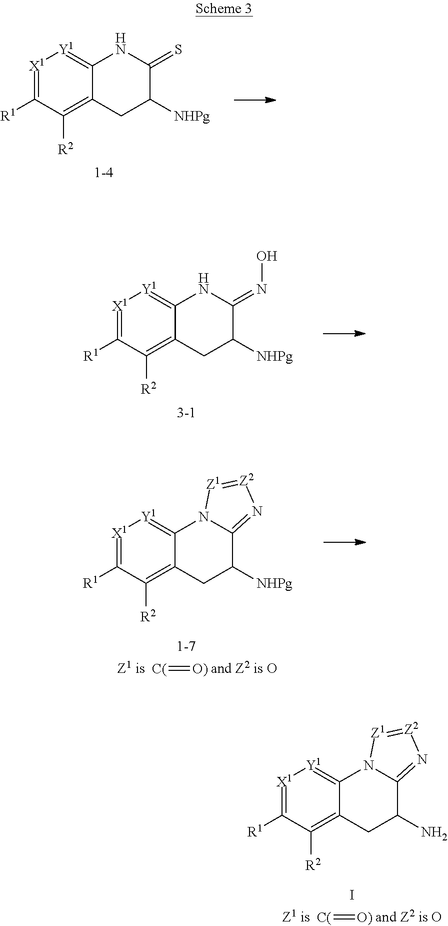

Scheme 4 refers to a preparation of compounds of Formula 1-7. Compounds of Formula 4-1 may be prepared as described in Schemes 1 and 2, by utilizing a starting material wherein R¹ is replaced by Br. A compound of Formula 1-7 can be prepared by heating a compound of Formula 4-1 with a boronic acid of Formula 4-2 in which R¹ can be, for example, optionally substituted phenyl or heteroaryl in the presence of a palladium catalyst [e.g., Pd(PPh₃)₄] and an appropriate base (e.g., Na₂CO₃) in a suitable solvent (e.g., ethanol) or by using alternate Suzuki coupling conditions well known to those skilled in the art [see N. Miyaura and A. Suzuki, Chem. Rev. 1995, 95, 2457-2483]. A compound of Formula 1-7 can be converted to a compound of Formula I using chemistry described in Scheme I.

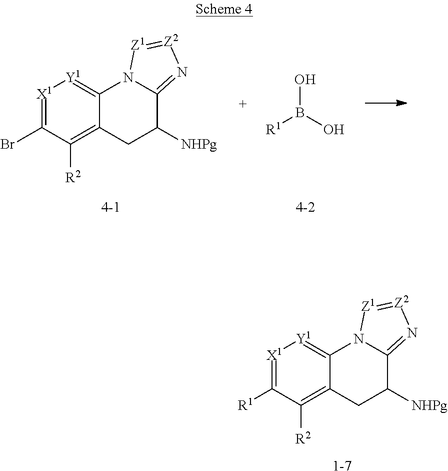

### EXAMPLES

Experiments were generally carried out under inert atmosphere (nitrogen or argon), particularly in cases where oxygen- or moisture-sensitive reagents or intermediates were employed. Commercial solvents and reagents were generally used without further purification, including anhydrous solvents where appropriate (generally Sure-Seal™ products from the Aldrich Chemical Company, Milwaukee, Wis.). Products were generally dried under vacuum before being carried on to further reactions or submitted for biological testing. Mass spectrometry data is reported from either liquid chromatography-mass spectrometry (LCMS), atmospheric pressure chemical ionization (APCI) or gas chromatography-mass spectrometry (GCMS) instrumentation. Chemical shifts for nuclear magnetic resonance (NMR) data are expressed in parts per million (ppm, δ) referenced to residual peaks from the deuterated solvents employed.

For syntheses referencing procedures in other Examples or Methods, reaction conditions (length of reaction and temperature) may vary. In general, reactions were followed by thin layer chromatography or mass spectrometry, and subjected to work-up when appropriate. Purifications may vary between experiments: in general, solvents and the solvent ratios used for eluents/gradients were chosen to provide appropriate R_{f}s or retention times.

### Example 1

### 4-Amino-7-(3-methoxyphenoxy)-4,5-dihydro[1,2,4]triazolo[4,3-a]quinolin-1(2H)-one ENT-1, hydrochloride salt (1)

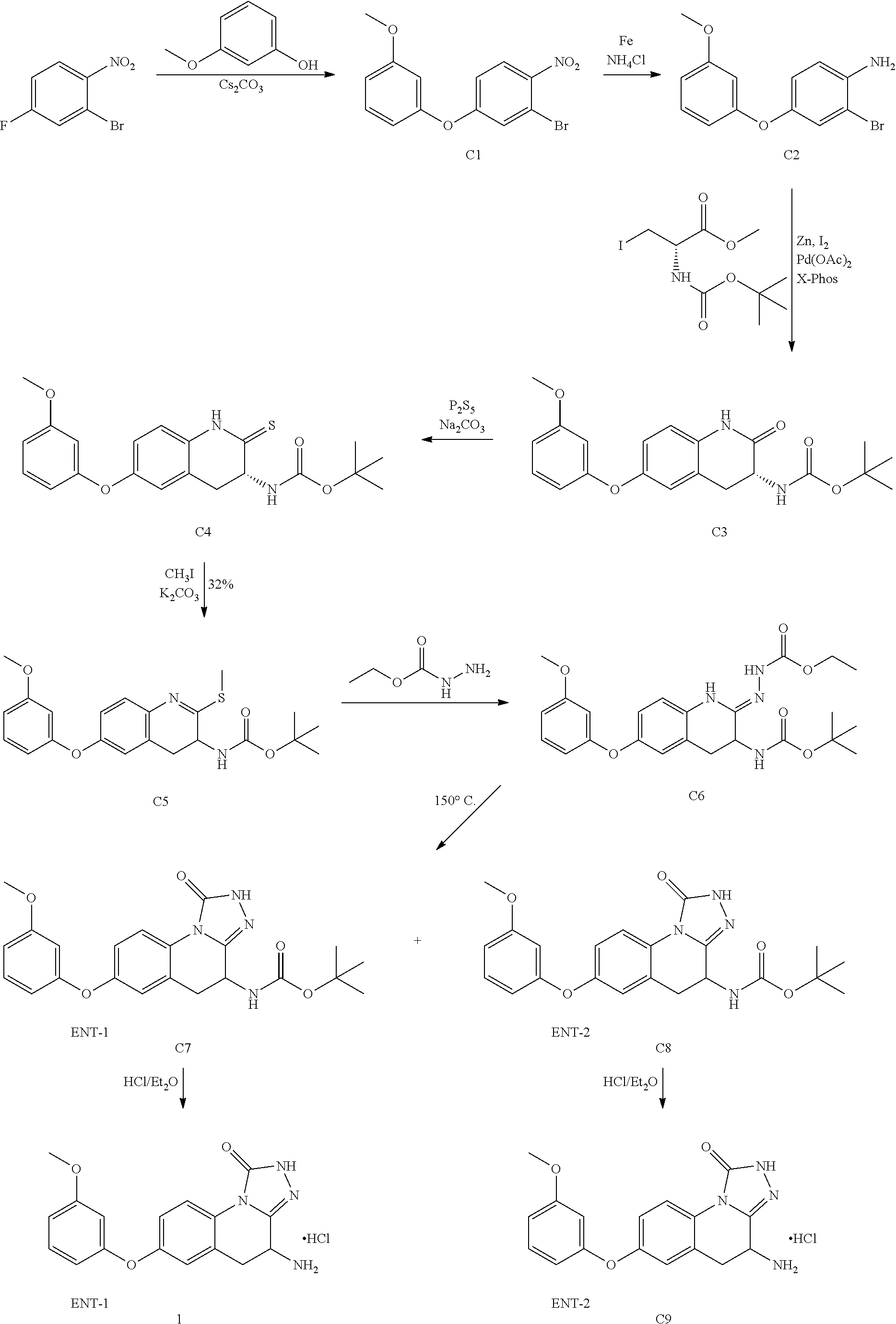

### Step 1. Synthesis of 2-bromo-4-(3-methoxyphenoxy)-1-nitrobenzene (C1)

To a 0° C. solution of 2-bromo-4-fluoro-1-nitrobenzene (20 g, 91 mmol) in acetonitrile (300 mL) was added cesium carbonate (36 g, 110 mmol) followed by 3-methoxyphenol (12.0 ml, 109 mmol), and the reaction mixture was stirred for 12 hours at room temperature. Solvent was removed in vacuo, and the residue was diluted with ethyl acetate and washed with water. The organic layer was dried over sodium sulfate, filtered, and concentrated under reduced pressure. Purification via silica gel chromatography (Eluent: 1% ethyl acetate in petroleum ether) afforded the product as a pale yellow liquid. Yield: 24.8 g, 76.5 mmol, 84%. GCMS m/z 323.1 [M⁺]. ¹H NMR (400 MHz, DMSO-d₆) δ 8.10 (d, J=9.1 Hz, 1H), 7.44 (d, J=2.6 Hz, 1H), 7.40 (dd, J=8.4, 8.1 Hz, 1H), 7.11 (dd, J=9.1, 2.6 Hz, 1H), 6.89 (br dd, J=8.4, 2.3 Hz, 1H), 6.80 (br dd, J=2.6, 2.3 Hz, 1H), 6.75 (br dd, J=7.9, 2.3 Hz, 1H), 3.77 (s, 3H).

### Step 2. Synthesis of 2-bromo-4-(3-methoxyphenoxy)aniline (C2)

Iron powder (26.2 g, 469 mmol) was added to a solution of 2-bromo-4-(3-methoxyphenoxy)-1-nitrobenzene (C1) (36 g, 110 mmol) in a 2:1:1 mixture of tetrahydrofuran, methanol and water (580 mL). Ammonium chloride (23.8 g, 445 mmol) was added and the reaction mixture was heated to 70° C. for 3 hours. After filtration through a pad of Celite, the reaction mixture was concentrated in vacuo to afford an aqueous residue, which was diluted with ethyl acetate and washed with water. The organic layer was dried over sodium sulfate, filtered, and concentrated under reduced pressure; trituration with diethyl ether provided the product as a brown solid. Yield: 29 g, 99 mmol, 90%. LCMS m/z 294.2 [M+H⁺]. ¹H NMR (300 MHz, DMSO-d₆) δ 7.21 (dd, J=8.4, 8.0 Hz, 1H), 7.09 (dd, J=2.1, 0.7 Hz, 1H), 6.86 (dd, half of ABX pattern, J=8.7, 2.1 Hz, 1H), 6.83 (dd, half of ABX pattern, J=8.7, 0.7 Hz, 1H), 6.63 (ddd, J=8.2, 2.4, 0.9 Hz, 1H), 6.46 (dd, J=2.4, 2.1 Hz, 1H), 6.42 (ddd, J=8.0, 2.4, 0.7 Hz, 1H), 5.20 (br s, 2H), 3.71 (s, 3H).

### Step 3. Synthesis of tert-butyl [(3R)-6-(3-methoxyphenoxy)-2-oxo-1,2,3,4-tetrahydroquinolin-3-yl]carbamate (C3)

Zinc (1.38 g, 21.1 mmol) was dried for 30 minutes under vacuum using a heat gun and then suspended in N,N-dimethylformamide (10 mL). Crystals of iodine (0.267 g, 1.05 mmol) were added, and the resulting deep red solution was stirred until the color disappeared. To this solution was added methyl N-(tert-butoxycarbonyl)-3-iodo-D-alaninate (6.26 g, 19.0 mmol) and stirring was continued for 30 minutes. In a separate flask, a mixture of palladium(II) acetate (47 mg, 0.21 mmol) and 2-dicyclohexylphosphino-2′,4′,6′-triisopropylbiphenyl (X-Phos, 0.252 g, 0.529 mmol) in N,N-dimethylformamide (15 mL) was stirred for 5 minutes before addition of 2-bromo-4-(3-methoxyphenoxy)aniline (C2) (3.1 g, 11 mmol). The zincate solution was added to this flask, and the reaction mixture was heated at 60° C. for 12 hours. After dilution with ethyl acetate, the reaction mixture was washed with ice-cold water. The organic layer was dried over sodium sulfate, filtered, and concentrated in vacuo; purification via chromatography on silica gel (Eluent: 20% ethyl acetate in petroleum ether) afforded the product as a brown solid. Yield: 2.3 g, 6.0 mmol, 55%. LCMS m/z 329.0 {[M−(2-methylprop-1-ene)]+H⁺}. ¹H NMR (400 MHz, DMSO-d₆) δ 10.23 (br s, 1H), 7.23 (dd, J=8.3, 8.1 Hz, 1H), 6.93-7.00 (br m, 2H), 6.85-6.88 (m, 2H), 6.66 (ddd, J=8.2, 2.3, 0.7 Hz, 1H), 6.52 (dd, J=2.4, 2.2 Hz, 1H), 6.48 (ddd, J=8.1, 2.4, 0.8 Hz, 1H), 4.10-4.21 (m, 1H), 3.72 (s, 3H), 2.86-3.01 (m, 2H), 1.40 (s, 9H).

### Step 4. Synthesis of tert-butyl [(3R)-6-(3-methoxyphenoxy)-2-thioxo-1,2,3,4-tetrahydroquinolin-3-yl]carbamate (C4)

Sodium carbonate (1.46 g 13.8 mmol) and phosphorus pentasulfide (3.06 g, 13.8 mmol) were combined in tetrahydrofuran (50 mL) and stirred for 30 minutes at room temperature. tert-Butyl [(3R)-6-(3-methoxyphenoxy)-2-oxo-1,2,3,4-tetrahydroquinolin-3-yl]carbamate (C3) (2.3 g, 6.0 mmol) was added and the reaction mixture was heated at reflux for 12 hours, then poured into ice water and extracted with ethyl acetate. The combined organic layers were washed with water and with saturated aqueous sodium chloride solution, dried over sodium sulfate, filtered, and concentrated in vacuo. Purification using silica gel chromatography (Eluent: 5% ethyl acetate in petroleum ether) provided the product as a yellow solid. Yield: 1.8 g, 4.5 mmol, 75%. LCMS m/z 401.1 [M+H⁺]. ¹H NMR (400 MHz, DMSO-d₆) δ 12.33 (br s, 1H), 7.26 (dd, J=8.3, 8.1 Hz, 1H), 7.11 (d, J=8.6 Hz, 1H), 7.04 (v br d, J=7.8 Hz, 1H), 6.97 (br d, J=2.7 Hz, 1H), 6.92 (dd, J=8.3, 2.7 Hz, 1H), 6.69 (ddd, J=8.3, 2.4, 0.8 Hz, 1H), 6.55 (dd, J=2.4, 2.2 Hz, 1H), 6.52 (ddd, J=8.1, 2.2, 0.7 Hz, 1H), 4.20-4.32 (m, 1H), 3.73 (s, 3H), 3.01 (dd, J=15.9, 5.9 Hz, 1H), 2.80 (br dd, J=15, 14 Hz, 1H), 1.41 (s, 9H).

### Step 5. Synthesis of tert-butyl [6-(3-methoxyphenoxy)-2-(methylsulfanyl)-3,4-dihydroquinolin-3-yl]carbamate (C5)

A solution of tert-butyl [(3R)-6-(3-methoxyphenoxy)-2-thioxo-1,2,3,4-tetrahydroquinolin-3-yl]carbamate (C4) (1.8 g, 4.5 mmol) in tetrahydrofuran (50 mL) was cooled to 15° C. Potassium carbonate (3.1 g, 22 mmol) was added, followed by methyl iodide (3.34 mL, 54.0 mmol), and the reaction mixture was stirred for 20 hours at 15° C. The reaction mixture was then diluted with ethyl acetate and washed with ice-cold water. The organic layer was dried over sodium sulfate, filtered, and concentrated under reduced pressure; the residue was purified using chromatography on silica gel (Eluent: 6% ethyl acetate in petroleum ether) to afford the product as a solid. Chiral analysis via HPLC [Column: Chiral Technologies Chiralpak IA, 5 μm; Eluent: 20% 2-propanol in (0.1% diethylamine in hexanes)] revealed that racemization occurred during this transformation. Yield: 0.60 g, 1.4 mmol, 31%. LCMS m/z 415.1 [M+H⁺]. ¹H NMR (400 MHz, DMSO-d₆) δ 7.48 (br d, J=9.3 Hz, 1H), 7.27 (dd, J=8.3, 8.1 Hz, 1H), 7.18-7.21 (m, 1H), 6.84-6.89 (m, 2H), 6.70 (ddd, J=8.3, 2.3, 0.9 Hz, 1H), 6.57 (dd, J=2.4, 2.2 Hz, 1H), 6.54 (ddd, J=8.1, 2.2, 0.7 Hz, 1H), 4.25-4.34 (m, 1H), 3.73 (s, 3H), 2.81 (d, J=10.5 Hz, 2H), 2.35 (s, 3H), 1.42 (s, 9H).

### Step 6. Synthesis of ethyl 2-{3-[(tert-butoxycarbonyl)amino]-6-(3-methoxyphenoxy)-3,4-dihydroquinolin-2(1H)-ylidene}hydrazinecarboxylate (C6)

Ethyl hydrazinecarboxylate (0.15 g, 1.4 mmol) was added to a solution of tert-butyl [6-(3-methoxyphenoxy)-2-(methylsulfanyl)-3,4-dihydroquinolin-3-yl]carbamate (C5) (0.60 g, 1.4 mmol) in ethanol (12 mL) and the reaction mixture was heated to reflux for 4 hours. After removal of solvent in vacuo, the crude residue was taken up in ethyl acetate and washed with ice-cold water. The organic layer was dried over sodium sulfate, filtered, and concentrated under reduced pressure; purification via silica gel chromatography (Eluent: 25% ethyl acetate in petroleum ether) provided the product as a solid. Yield: 0.29 g, 0.62 mmol, 44%. LCMS m/z 471.1 [M+H⁺]. ¹H NMR (400 MHz, DMSO-d₆) δ 8.96 (s, 1H), 7.23 (dd, J=8.3, 8.1 Hz, 1H), 6.83-6.90 (m, 3H), 6.65 (ddd, J=8.3, 2.4, 0.7 Hz, 1H), 6.50 (dd, J=2.4, 2.2 Hz, 1H), 6.47 (ddd, J=8.1, 2.2, 0.7 Hz, 1H), 4.19-4.28 (m, 1H), 4.10 (q, J=7.1 Hz, 2H), 3.72 (s, 3H), 2.97 (dd, J=15.6, 4.4 Hz, 1H), 2.77 (dd, J=15.6, 9.5 Hz, 1H), 1.39 (s, 9H), 1.22 (t, J=7.0 Hz, 3H).

### Step 7. Synthesis of tert-butyl [7-(3-methoxyphenoxy)-1-oxo-1,2,4,5-tetrahydro[1,2,4]triazolo[4,3-a]quinolin-4-yl]carbamate ENT-1 (C7) and tert-butyl [7-(3-methoxyphenoxy)-1-oxo-1,2,4,5-tetrahydro[1,2,4]triazolo[4,3-a]quinolin-4-yl]carbamate ENT-2 (C8)

A solution of ethyl 2-{3-[(tert-butoxycarbonyl)amino]-6-(3-methoxyphenoxy)-3,4-dihydroquinolin-2(1H)-ylidene}hydrazinecarboxylate (C6) (0.25 g, 0.53 mmol) in N,N-dimethylformamide (5 mL) was heated to 150° C. for 2 hours. The reaction mixture was poured into ice-cold water and extracted with ethyl acetate; the organic layer was then dried over sodium sulfate, filtered, and concentrated in vacuo. Purification via silica gel chromatography (Eluent: 25% ethyl acetate in petroleum ether) provided the racemic product tert-butyl [7-(3-methoxyphenoxy)-1-oxo-1,2,4,5-tetrahydro[1,2,4]triazolo[4,3-a]quinolin-4-yl]carbamate. Yield: 0.12 g, 0.28 mmol, 53%. Material derived from another run of this procedure (0.20 g, 0.47 mmol) was separated into its enantiomers via chiral HPLC (Column: Chiral Technologies Chiralpak®-IA, 5 μm; Eluent: 15% ethanol in hexanes containing 0.1% diethylamine), to afford the first-eluting isomer tert-butyl [7-(3-methoxyphenoxy)-1-oxo-1,2,4,5-tetrahydro[1,2,4]triazolo[4,3-a]quinolin-4-yl]carbamate ENT-1 (C7) as a solid. Yield: 50 mg, 0.12 mmol, 26%. LCMS m/z 425.0 [M+H⁺]. ¹H NMR (400 MHz, DMSO-d₆) δ 11.91 (s, 1H), 8.21 (d, J=8.8 Hz, 1H), 7.41-7.48 (m, 1H), 7.28 (dd, J=8.3, 7.8 Hz, 1H), 7.09 (d, J=2.9 Hz, 1H), 7.03 (dd, J=8.8, 2.4 Hz, 1H), 6.72 (dd, J=7.8, 2.4 Hz, 1H), 6.58 (dd, J=2.4, 2.0 Hz, 1H), 6.55 (dd, J=8, 2 Hz, 1H), 4.75-4.85 (m, 1H), 3.74 (s, 3H), 3.11 (dd, half of ABX pattern, J=15.6, 5.4 Hz, 1H), 2.98 (dd, half of ABX pattern, J=15.6, 9.8 Hz, 1H), 1.40 (s, 9H).

Also obtained was the second-eluting enantiomer tert-butyl [7-(3-methoxyphenoxy)-1-oxo-1,2,4,5-tetrahydro[1,2,4]triazolo[4,3-a]quinolin-4-yl]carbamate ENT-2 (C8) as a solid. Yield: 100 mg, 0.236 mmol, 50%. LCMS m/z 425.0 [M+H⁺]. ¹H NMR (400 MHz, DMSO-d₆) δ 11.91 (s, 1H), 8.21 (d, J=8.8 Hz, 1H), 7.41-7.48 (m, 1H), 7.28 (dd, J=8.3, 8.3 Hz, 1H), 7.08-7.10 (m, 1H), 7.03 (dd, J=9, 3 Hz, 1H), 6.72 (br dd, J=8, 2 Hz, 1H), 6.58 (dd, J=2.4, 2.2 Hz, 1H), 6.55 (br dd, J=8, 2 Hz, 1H), 4.74-4.85 (m, 1H), 3.74 (s, 3H), 3.11 (dd, half of ABX pattern, J=16, 6 Hz, 1H), 2.98 (dd, half of ABX pattern, J=16, 9 Hz, 1H), 1.40 (s, 9H).

### Step 8. Synthesis of 4-amino-7-(3-methoxyphenoxy)-4,5-dihydro[1,2,4]triazolo[4,3-a]quinolin-1(2H)-one ENT-1, hydrochloride salt (1)

tert-Butyl [7-(3-methoxyphenoxy)-1-oxo-1,2,4,5-tetrahydro[1,2,4]triazolo[4,3-a]quinolin-4-yl]carbamate ENT-1 (C7) (50 mg, 0.12 mmol) was dissolved in diethyl ether (2 mL), cooled to 0° C., and treated with a solution of hydrogen chloride in diethyl ether (4 M, 5 mL). After the reaction mixture had been stirred at room temperature for 30 minutes, it was concentrated in vacuo, and the residue was triturated with pentane to provide the product (1) as a solid. Yield: 30 mg, 0.083 mmol, 69%. LCMS m/z 325.1 [M+H⁺]. ¹H NMR (400 MHz, DMSO-d₆) δ 12.32 (s, 1H), 8.84 (br s, 3H), 8.24 (d, J=8.8 Hz, 1H), 7.30 (dd, J=8.3, 8.1 Hz, 1H), 7.17 (br d, J=2.7 Hz, 1H), 7.10 (dd, J=8.8, 2.9 Hz, 1H), 6.74 (ddd, J=8.3, 2.4, 1.0 Hz, 1H), 6.58 (dd, J=2.4, 2.2 Hz, 1H), 6.56 (ddd, J=8.1, 2.2, 0.7 Hz, 1H), 4.78 (dd, J=9.3, 5.9 Hz, 1H), 3.74 (s, 3H), 3.3-3.40 (m, 1H, assumed; partially obscured by water peak), 3.15 (dd, J=16.0, 9.4 Hz, 1H).

In the same manner, tert-butyl [7-(3-methoxyphenoxy)-1-oxo-1,2,4,5-tetrahydro[1,2,4]triazolo[4,3-a]quinolin-4-yl]carbamate ENT-2 (C8) was converted to 4-amino-7-(3-methoxyphenoxy)-4,5-dihydro[1,2,4]triazolo[4,3-a]quinolin-1(2H)-one ENT-2, hydrochloride salt (C9), which was obtained as a solid. Yield: 67 mg, 0.19 mmol, 79%. LCMS m/z 325.1 [M+H⁺]. ¹H NMR (400 MHz, DMSO-d₆) δ 12.32 (s, 1H), 8.84 (br s, 3H), 8.24 (d, J=8.8 Hz, 1H), 7.30 (dd, J=8.3, 8.1 Hz, 1H), 7.17 (br d, J=2.7 Hz, 1H), 7.10 (dd, J=8.8, 2.7 Hz, 1H), 6.74 (ddd, J=8.3, 2.3, 0.9 Hz, 1H), 6.58 (dd, J=2.2, 2.2 Hz, 1H), 6.56 (ddd, J=8.0, 2.3, 0.9 Hz, 1H), 4.77 (dd, J=9.4, 5.7 Hz, 1H), 3.74 (s, 3H), 3.3-3.40 (m, 1H, assumed; partially obscured by water peak), 3.15 (dd, J=15.9, 9.3 Hz, 1H).

### Example 2

### 6-Amino-3-phenoxy-6,8-dihydro[1,2,4]triazolo[4,3-a][1,8]naphthyridin-9(5H)-one, ENT-1 (2)

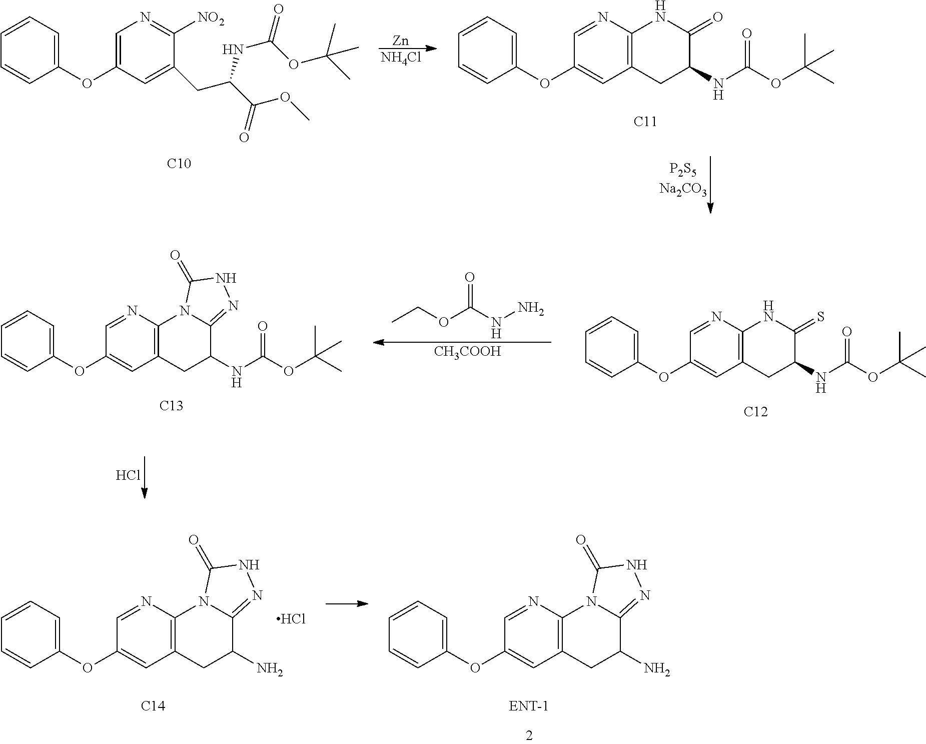

### Step 1. Synthesis of tert-butyl [(3S)-2-oxo-6-phenoxy-1,2,3,4-tetrahydro-1,8-naphthyridin-3-yl]carbamate (C11)

Zinc (1.88 g, 28.7 mmol) and ammonium chloride (3.08 g, 57.6 mmol) were added to a solution of methyl {(2S)-2-[(tert-butoxycarbonyl)amino]-3-(2-nitro-5-phenoxypyridin-3-yl)}propanoate (C10) (prepared according to M. M. Claffey et al., PCT Int. Appl. 2010, WO 2010146488 A1, Dec. 23, 2010) (1.20 g, 2.87 mmol) in tetrahydrofuran (4 mL) and methanol (8 mL), and the resulting slurry was heated at 60° C. for 48 hours. The reaction mixture was then treated with saturated aqueous sodium carbonate solution (15 mL) and ethyl acetate (100 mL), and allowed to stir for 10 minutes. The mixture was filtered, and the organic layer was washed with water (2×100 mL) and with saturated aqueous sodium chloride solution (100 mL), dried over sodium sulfate, filtered, and concentrated in vacuo. Purification via silica gel chromatography (Gradient: 0% to 70% ethyl acetate in heptane) afforded the product as a white foam. Yield: 750 mg, 2.11 mmol, 73%. This material contained a contaminant identified by NMR and MS as tert-butyl [(3S)-6-fluoro-2-oxo-1,2,3,4-tetrahydro-1,8-naphthyridin-3-yl]carbamate. LCMS m/z 356.2 [M+H⁺]. ¹H NMR (400 MHz, CDCl₃), product peaks only: δ 9.66 (br s, 1H), 8.08 (br d, J=2.5 Hz, 1H), 7.37 (br dd, J=8, 8 Hz, 2H), 7.22 (br d, J=2 Hz, 1H), 7.15 (br dd, J=8, 8 Hz, 1H), 7.00 (br d, J=8 Hz, 2H), 5.70 (br s, 1H) 4.32-4.42 (m, 1H), 3.45-3.54 (m, 1H), 2.75-2.85 (m, 1H), 1.47 (s, 9H).

### Step 2. Synthesis of tert-butyl [(3S)-6-phenoxy-2-thioxo-1,2,3,4-tetrahydro-1,8-naphthyridin-3-yl]carbamate (C12)

Sodium carbonate (99.5%, 673 mg, 6.32 mmol) and phosphorus pentasulfide (99%, 1.42 g, 6.32 mmol) were added to tetrahydrofuran (4.2 mL), and the suspension was vigorously stirred for 15 minutes at room temperature. To the resulting yellow solution was added a solution of tert-butyl [(3S)-2-oxo-6-phenoxy-1,2,3,4-tetrahydro-1,8-naphthyridin-3-yl]carbamate (C11) (from the preceding step, 748 mg, 2.10 mmol) in tetrahydrofuran (3 mL), and the reaction mixture was heated at 70° C. for 1 hour. After cooling to room temperature, the reaction mixture was poured into water and extracted with ethyl acetate. The combined organic layers were washed with water and with saturated aqueous sodium chloride solution, dried over magnesium sulfate, filtered, and concentrated in vacuo. Purification via silica gel chromatography (Gradient: 0% to 70% ethyl acetate in heptane) afforded the product as a yellow solid. Yield: 590 mg, 1.59 mmol, 76%. This material contained a contaminant identified by NMR and MS as tert-butyl [(3S)-6-fluoro-2-thioxo-1,2,3,4-tetrahydro-1,8-naphthyridin-3-yl]carbamate. LCMS m/z 372.2 [M+H⁺]. ¹H NMR (400 MHz, CDCl₃), product peaks only: δ 11.57 (br s, 1H), 8.33 (dd, J=2.7, 0.8 Hz, 1H), 7.40 (br dd, J=8.5, 7.5 Hz, 2H), 7.17-7.23 (m, 2H), 7.02-7.06 (m, 2H), 6.21 (br s, 1H), 4.34-4.42 (m, 1H), 3.45-3.55 (m, 1H), 2.68-2.79 (m, 1H), 1.49 (s, 9H).

### Step 3. Synthesis of tert-butyl (9-oxo-3-phenoxy-5,6,8,9-tetrahydro[1,2,4]triazolo[4,3-a][1,8]naphthyridin-6-yl)carbamate (C13)

Ethyl hydrazinecarboxylate (168 mg, 1.61 mmol), magnesium sulfate (100 mg) and acetic acid (31 uL, 0.54 mmol) were added to a solution of tert-butyl [(3S)-6-phenoxy-2-thioxo-1,2,3,4-tetrahydro-1,8-naphthyridin-3-yl]carbamate (C12) (200 mg, 0.538 mmol) in cyclohexanol (2.7 mL), and the reaction mixture was heated to 160° C. for 90 minutes. After addition of ethyl hydrazinecarboxylate (0.5 equivalents) and acetic acid (0.10 mL), heating was continued for 1 hour. Additional acetic acid (0.10 mL) was introduced, and the reaction mixture was heated for an additional 2.25 hours, then cooled to room temperature. Celite was added, and solvents were removed in vacuo; purification via silica gel chromatography using the Celite mixture as a pre-column (Gradient: 0% to 10% methanol in dichloromethane) afforded the product as a yellow foam. Yield: 175 mg, 0.443 mmol, 82%. Chiral HPLC evaluation of a related compound from a similar reaction revealed extensive loss of stereochemical integrity after this transformation; for this reason, products of this reaction type were assumed to be racemic. LCMS m/z 396.1 [M+H⁺]. ¹H NMR (400 MHz, CDCl₃), characteristic peaks: δ 8.33 (br d, J=2.7 Hz, 1H), 7.40 (br dd, J=8.6, 7.6 Hz, 2H), 7.25 (br d, J=2.7 Hz, 1H), 7.20 (tt, J=7.4, 1.1 Hz, 1H), 7.04 (br dd, J=8.7, 1.1 Hz, 2H), 3.32-3.40 (m, 1H), 2.87-2.97 (m, 1H), 1.48 (s, 9H).

### Step 4. Synthesis of 6-amino-3-phenoxy-6,8-dihydro[1,2,4]triazolo[4,3-a][1,8]naphthyridin-9(5H)-one, hydrochloride salt (C14)

tert-Butyl (9-oxo-3-phenoxy-5,6,8,9-tetrahydro[1,2,4]triazolo[4,3-a][1,8]naphthyridin-6-yl)carbamate (C13) (175 mg, 0.443 mmol) was mixed with a solution of hydrogen chloride in 2-propanol (5-6 M, 6 mL), and the reaction mixture was allowed to stir for 1.25 hours. After addition of Celite (1 g), solvent was removed in vacuo, and purification was carried out via silica gel chromatography using the Celite mixture as a pre-column [Gradient: 0% to 10% (10% concentrated ammonium hydroxide in methanol) in dichloromethane]. The resulting material was converted to its hydrochloride salt as follows: the solid was suspended in a solution of hydrogen chloride in diethyl ether (1 M), then solvent was removed in vacuo. This procedure was repeated, and the residue was suspended twice in diethyl ether (1 mL), followed by concentration under reduced pressure, to afford the product as a white foam. Yield: 68 mg, 0.20 mmol, 45%. LCMS m/z 296.0 [M+H⁺]. ¹H NMR (400 MHz, CD₃OD) δ 8.13 (br d, J=2.7 Hz, 1H), 7.56 (br d, J=2.8 Hz, 1H), 7.43 (br dd, J=8.6, 7.5 Hz, 2H), 7.20-7.25 (m, 1H), 7.08-7.12 (m, 2H), 4.83 (dd, J=9.7, 5.9 Hz, 1H), 3.48 (dd, J=16.0, 5.9 Hz, 1H), 3.24 (br dd, J=16.0, 9.8 Hz, 1H).

### Step 5. Synthesis of 6-amino-3-phenoxy-6,8-dihydro[1,2,4]triazolo[4,3-a][1,8]naphthyridin-9(5H)-one ENT-1 (2)

Chiral separation of 6-amino-3-phenoxy-6,8-dihydro[1,2,4]triazolo[4,3-a][1,8]naphthyridin-9(5H)-one, hydrochloride salt (C14) was carried out via supercritical fluid chromatography (Column: Chiral Technologies Chiralcel OJ-H, 5 μm; Eluent: 4:1 carbon dioxide/ethanol containing 0.2% isopropylamine). The first-eluting enantiomer was the product, obtained as a gum. Retention time: 4.92 minutes (Column: Chiral Technologies Chiralcel OJ-H, 5 μm, 4.6×25 mm; Eluent: 4:1 carbon dioxide/ethanol containing 0.2% isopropylamine; Flow rate 2.5 ml/min). LCMS m/z 296.1 [M+H⁺].

### Example 3

### 4-Amino-7-[3-(trifluoromethyl)phenoxyl-4,5-dihydro[1,2,4]triazolo[1,3-a]quinolin-1(2H)-one, hydrochloride salt (3)

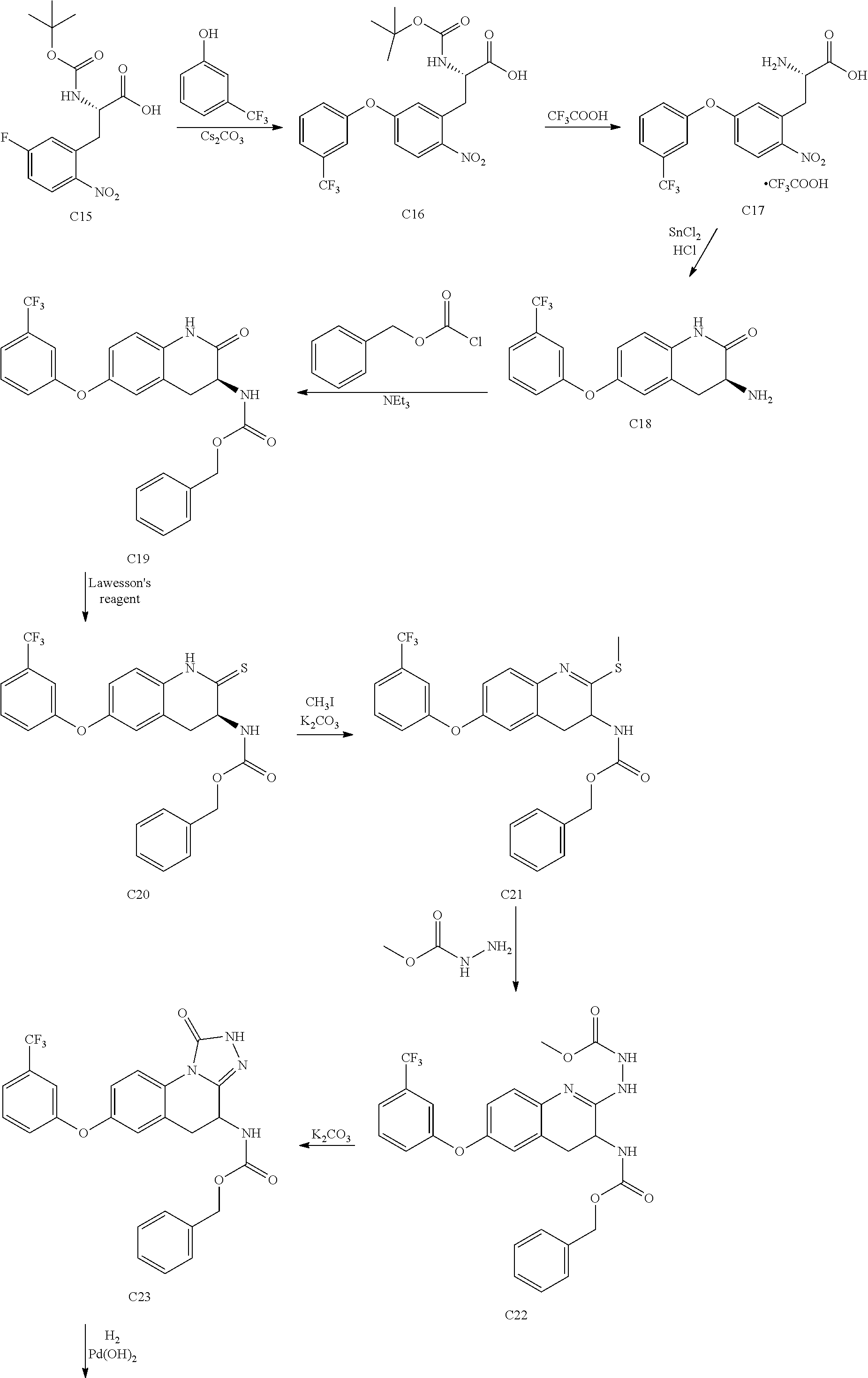

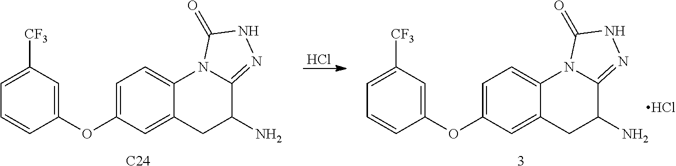

### Step 1. Synthesis of (2S)-2-[(tert-butoxycarbonyl)amino]-3-{2-nitro-5-[3-(trifluoromethyl)phenoxy]phenyl}propanoic acid (C16)

The product was prepared from (2S)-2-[(tertbutoxycarbonyl)amino]-3-(5-fluoro-2-nitrophenyl)propanoic acid (C15) (prepared according to M. M. Claffey et al., PCT Int. Appl. 2010, WO 2010146488 A1, Dec. 23, 2010) and 3-(trifluoromethyl)phenol according to the general procedure for the synthesis of 2-bromo-4-(3-methoxyphenoxy)-1-nitrobenzene (C1) in Example 1. In this case the eluent used for chromatography was 2% methanol in dichloromethane. Yield: 5.0 g, 11 mmol, 65%. LCMS m/z 371.0 [(M-BOC)+H⁺]. ¹H NMR (300 MHz, DMSO-d₆) δ 8.06 (d, J=9.1 Hz, 1H), 7.59-7.74 (m, 2H), 7.38-7.50 (m, 2H), 7.18 (br d, J=2.8 Hz, 1H), 7.05 (br dd, J=9.1, 2.4 Hz, 1H), 6.75-6.94 (br m, 1H), 4.12-4.28 (br m, 1H), 3.54 (dd, J=13.6, 4.2 Hz, 1H), 2.95 (dd, J=13.2, 11.0 Hz, 1H), 1.27 (s, 9H).

### Step 2. Synthesis of (2S)-2-amino-3-{2-nitro-5-[3-(trifluoromethyl)phenoxy]phenyl}propanoic acid, trifluoroacetate salt (C17)

To a stirring solution of (2S)-2-[(tert-butoxycarbonyl)amino]-3-{2-nitro-5-[3-(trifluoromethyl)phenoxy]phenyl}propanoic acid (C16) (5.5 g, 12 mmol) in dichloromethane (55 mL) was added trifluoroacetic acid (55 mL) and the reaction mixture was stirred at room temperature for 3 hours. Solvent was removed via distillation, and the residue was triturated with n-pentane to provide the product, which was a 1:1 mixture with ethyl acetate by ¹H NMR analysis. Corrected yield: 3.88 g, 10.5 mmol, 88%. This material was taken to the following step without further purification. LCMS m/z 371.0 [M+H⁺]. ¹H NMR (300 MHz, DMSO-d₆) δ 8.37 (br s, 3H), 8.20 (d, J=9.1 Hz, 1H), 7.60-7.77 (m, 2H), 7.54 (br s, 1H), 7.49 (br d, J=7.7 Hz, 1H), 7.23 (br s, 1H), 7.17 (br d, J=9.1 Hz, 1H), 4.16-4.30 (m, 1H), 3.64 (dd, J=13.9, 6.3 Hz, 1H), 3.19 (dd, J=13.9, 8.4 Hz, 1H).

### Step 3. Synthesis of (3S)-3-amino-6-[3-(trifluoromethyl)phenoxy]-3,4-dihydroquinolin-2(1H)-one (C18)

To a mixture of (2S)-2-amino-3-{2-nitro-5-[3-(trifluoromethyl)phenoxy]phenyl}propanoic acid, trifluoroacetate salt (C17) (3.88 g from the preceding step, 10.5 mmol), methanol (50 mL) and a saturated solution of hydrogen chloride in methanol (50 mL) was added tin(II) chloride dihydrate (18.2 g, 80.7 mmol). The reaction mixture was heated to reflux for 2 hours, then allowed to cool to room temperature and quenched with water. After basification with aqueous sodium bicarbonate solution, the mixture was extracted with ethyl acetate. The combined organic layers were dried over sodium sulfate, filtered, and concentrated under reduced pressure to obtain the product (5.0 g), which was taken directly to the following step without additional purification.

### Step 4. Synthesis of benzyl {(3S)-2-oxo-6-[3-(trifluoromethyl)phenoxy]-1,2,3,4-tetrahydroquinolin-3-yl}carbamate (C19)

(3S)-3-Amino-6-[3-(trifluoromethyl)phenoxy]-3,4-dihydroquinolin-2(1H)-one (C18) (5.0 g from the preceding step, ≦10.5 mmol) and benzyl chloroformate (4.3 mL, 30 mmol) were combined in dichloromethane (50 mL) and cooled to 10° C. Triethylamine (4.4 mL, 32 mmol) was added, and the reaction mixture was stirred at 10° C. for 4 hours. The reaction mixture was then allowed to warm to room temperature and quenched by addition of water. After extraction with ethyl acetate, the combined organic layers were dried over sodium sulfate, filtered, and concentrated in vacuo to obtain the product (4.6 g, ≦10 mmol), which was taken to the following step without purification. LCMS m/z 457.2 [M+H⁺].

### Step 5. Synthesis of benzyl {(3S)-2-thioxo-6-[3-(trifluoromethyl)phenoxy]-1,2,3,4-tetrahydroquinolin-3-yl}carbamate (C20)

To a solution of benzyl {(3S)-2-oxo-6-[3-(trifluoromethyl)phenoxy]-1,2,3,4-tetrahydroquinolin-3-yl}carbamate (C19) (4.6 g from the preceding step, ≦10 mmol) in toluene (138 mL) was added Lawesson's reagent [2,4-bis(4-methoxyphenyl)-1,3,2,4-dithiasiphosphetane-2,4-dithione] (6.1 g, 15 mmol). The reaction mixture was heated to reflux for 3 hours, then cooled and concentrated in vacuo. Purification via silica gel chromatography (Eluent: 15% ethyl acetate in petroleum ether) provided the product. Yield: 0.91 g, 1.9 mmol, 16% over four steps. LCMS m/z 473.0 [M+H⁺]. ¹H NMR (400 MHz, DMSO-d₆), characteristic peaks: δ 12.40 (s, 1H), 7.60 (dd, J=8.1, 7.9 Hz, 1H), 7.47 (br d, J=7.7 Hz, 1H), 7.28-7.31 (m, 1H), 7.26 (br dd, J=8.1, 2.6 Hz, 1H), 7.16 (d, J=8.6 Hz, 1H), 7.05-7.09 (m, 1H), 7.00 (dd, J=8.6, 2.8 Hz, 1H), 5.08 (s, 2H), 4.35-4.44 (m, 1H), 3.05 (dd, J=16.0, 6.0 Hz, 1H), 2.87 (br dd, J=15, 13 Hz, 1H).

### Step 6. Synthesis of benzyl {2-(methylsulfanyl)-6-[3-(trifluoromethyl)phenoxy]-3,4-dihydroquinolin-3-yl}carbamate (C21)

The product was prepared from benzyl {(3S)-2-thioxo-6-[3-(trifluoromethyl)phenoxy]-1,2,3,4-tetrahydroquinolin-3-yl}carbamate (C20) according to the general procedure for the synthesis of tert-butyl [6-(3-methoxyphenoxy)-2-(methylsulfanyl)-3,4-dihydroquinolin-3-yl]carbamate (C5) in Example 1. In this case, the reaction mixture was simply filtered and concentrated in vacuo to afford the product. Yield: 0.80 g, 1.6 mmol, 84%. LCMS m/z 487.2 [M+H⁺]. ¹H NMR (300 MHz, DMSO-d₆) δ 7.97 (d, J=9.1 Hz, 1H), 7.61 (dd, J=8.0, 7.7 Hz, 1H), 7.47 (br d, J=7.7 Hz, 1H), 7.22-7.42 (m, 8H), 6.92-7.00 (m, 2H), 5.09 (s, 2H), 4.32-4.45 (m, 1H), 2.83-2.93 (m, 2H), 2.37 (s, 3H).

### Step 7. Synthesis of methyl 2-(3-{[(benzyloxy)carbonyl]amino}-6-[3-(trifluoromethyl)phenoxy]-3,4-dihydroquinolin-2-yl)hydrazinecarboxylate (C22)

To a solution of benzyl {2-(methylsulfanyl)-6-[3-(trifluoromethyl)phenoxy]-3,4-dihydroquinolin-3-yl}carbamate (C21) (0.80 g, 1.6 mmol) in methanol (15 mL) was added methyl hydrazinecarboxylate (0.15 g, 1.7 mmol) and the reaction mixture was heated to reflux for 24 hours. The resulting solid was isolated via filtration and triturated with n-pentane to afford the product. Yield: 0.310 g, 0.587 mmol, 37%. ¹H NMR (400 MHz, DMSO-d₆) δ 9.31-9.46 (br s, 1H), 9.04 (s, 1H), 7.57 (br dd, J=8, 8 Hz, 1H), 7.45-7.52 (br m, 1H), 7.42 (br d, J=8 Hz, 1H), 7.27-7.39 (m, 5H), 7.19-7.24 (m, 2H), 6.95-6.99 (br s, 1H), 6.93 (dd, half of ABX pattern, J=8.6, 2.4 Hz, 1H), 6.89 (d, half of AB quartet, J=8.6 Hz, 1H), 4.99-5.09 (m, 2H), 4.32-4.40 (m, 1H), 3.65 (s, 3H), 2.99 (dd, J=15.8, 4.6 Hz, 1H), 2.86 (dd, J=15.8, 8.8 Hz, 1H).

### Step 8. Synthesis of benzyl {1-oxo-7-[3-(trifluoromethyl)phenoxy]-1,2,4,5-tetrahydro[1,2,4]triazolo[4,3-a]quinolin-4-yl}carbamate (C23)

Potassium carbonate (3.4 g, 25 mmol) was added to a solution of methyl 2-(3-{[(benzyloxy)carbonyl]amino}-6-[3-(trifluoromethyl)phenoxy]-3,4-dihydroquinolin-2-yl)hydrazinecarboxylate (C22) (0.26 g, 0.49 mmol) in acetonitrile (20 mL), and the reaction mixture was heated to 100° C. for 1 hour in a microwave reactor. After removal of solvent in vacuo, the residue was quenched with water and extracted with ethyl acetate. The combined organic layers were dried over sodium sulfate, filtered, and concentrated under reduced pressure to yield a residue, which was triturated with hexanes to afford the product. Yield: 227 mg, 0.457 mmol, 93%. LCMS m/z 497.2 [M+H⁺].

### Step 9. Synthesis of 4-amino-7-[3-(trifluoromethyl)phenoxy]-4,5-dihydro[1,2,4]triazolo[4,3-a]quinolin-1(2H)-one (C24)

A solution of benzyl {1-oxo-7-[3-(trifluoromethyl)phenoxy]-1,2,4,5-tetrahydro[1,2,4]triazolo[4,3-a]quinolin-4-yl}carbamate (C23) (0.270 g, 0.544 mmol) in anhydrous ethanol was degassed with nitrogen for 10 minutes. Palladium hydroxide was added, and the reaction mixture was hydrogenated at 10 psi under hydrogen for 1 hour. The reaction was filtered through Celite and the filtrate was concentrated in vacuo. Silica gel chromatographic purification (Eluent: 5% methanol in dichloromethane) afforded the product. Yield: 110 mg, 0.304 mmol, 56%. LCMS m/z 363.2 [M+H⁺]. ¹H NMR (400 MHz, DMSO-d₆) δ 11.81-11.86 (br s, 1H), 8.26 (d, J=8.8 Hz, 1H), 7.62 (dd, J=7.9, 7.9 Hz, 1H), 7.49 (br d, J=7.9 Hz, 1H), 7.27-7.33 (m, 2H), 7.16 (d, J=2.8 Hz, 1H), 7.10 (dd, J=8.8, 2.8 Hz, 1H), 4.06 (dd, J=8.4, 5.1 Hz, 1H), 3.09 (dd, J=15.9, 5.2 Hz, 1H), 2.85 (dd, J=15.8, 8.4 Hz, 1H).

### Step 10. Synthesis of 4-amino-7-[3-(trifluoromethyl)phenoxy]-4,5-dihydro[1,2,4]triazolo[4,3-a]quinolin-1(2H)-one, hydrochloride salt (3)

4-Amino-7-[3-(trifluoromethyl)phenoxy]-4,5-dihydro[1,2,4]triazolo[4,3-a]quinolin-1(2H)-one (C24) (110 mg, 0.304 mmol) was dissolved in a 0° C. solution of hydrogen chloride in methanol (2 N, 11 ml), and the reaction mixture was stirred at 0° C. for 1 hour. Removal of solvent in vacuo provided a residue, which was triturated with diethyl ether to afford the product. Yield: 71 mg, 0.18 mmol, 59%. LCMS m/z 362.9 [M+H⁺]. ¹H NMR (300 MHz, DMSO-d₆) δ 12.33-12.35 (br s, 1H), 8.76-8.90 (br s, 3H), 8.29 (d, J=9.1 Hz, 1H), 7.64 (dd, J=8.7, 7.7 Hz, 1H), 7.51 (br d, J=8 Hz, 1H), 7.27-7.34 (m, 3H), 7.19 (dd, J=9.1, 2.8 Hz, 1H), 4.79 (dd, J=9.2, 6.1 Hz, 1H), 3.38 (dd, J=15.7, 5.9 Hz, 1H), 3.16 (dd, J=15.7, 9.4 Hz, 1H).

### Examples 4 and 5

### 7-(3-Methoxyphenoxy)-4,5-dihydro[1,2,4]triazolo[4,3-a]quinolin-4-amine, ENT-1 (4) and 7-(3-Methoxyphenoxy)-4,5-dihydro[1,2,4]triazolo[4,3-a]quinolin-4-amine, ENT-2 (5)

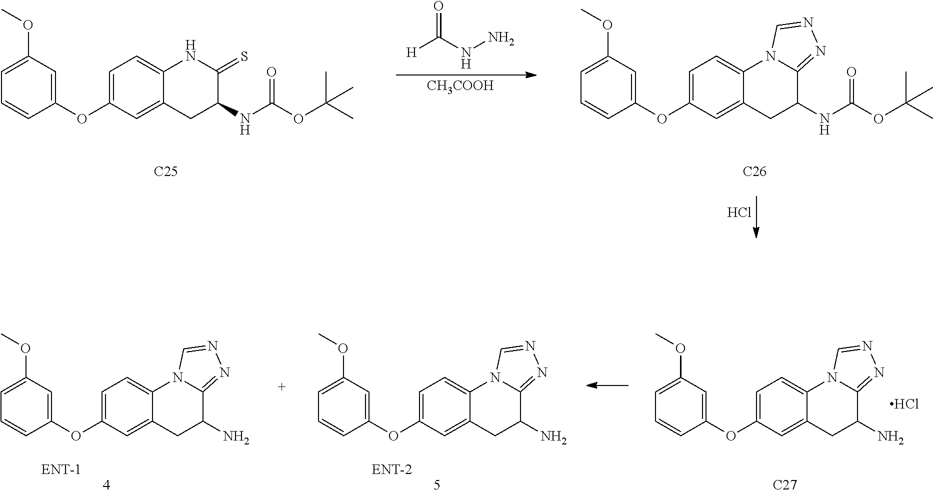

### Step 1. Synthesis of tert-butyl [7-(3-methoxyphenoxy)-4,5-dihydro[1,2,4]triazolo[4,3-a]quinolin-4-yl]carbamate (C26)

tert-Butyl [(3S)-6-(3-methoxyphenoxy)-2-thioxo-1,2,3,4-tetrahydroquinolin-3-yl]carbamate (C25, which was prepared in an analogous manner to its enantiomer C4 in Example 1, except that methyl N-(tert-butoxycarbonyl)-3-iodo-L-alaninate was used in place of its antipode) (500 mg, 1.25 mmol), formic hydrazide (200 mg, 3.33 mmol) and acetic acid (72 μL, 1.25 mmol) were combined in cyclohexanol (2 mL) and heated to 150° C. for 1 hour. The reaction mixture was allowed to cool, and cyclohexanol was removed by heating under high vacuum. The residue was purified via silica gel chromatography (Eluents: 50% ethyl acetate in heptane, followed by 5% methanol in ethyl acetate), affording the product as a colorless foam. Yield: 225 mg, 0.551 mmol, 44%. The product was assumed to have racemized at this step, as the product of the subsequent reaction was racemic. LCMS m/z 409.2 [M+H⁺]. ¹H NMR (400 MHz, CD₃OD) δ 9.14 (s, 1H), 7.70 (d, J=8.8 Hz, 1H), 7.25-7.30 (m, 1H), 7.09 (br d, J=2.5 Hz, 1H), 7.04 (dd, J=8.8, 2.7 Hz, 1H), 6.72-6.76 (m, 1H), 6.58-6.62 (m, 2H), 5.22 (dd, J=9.1, 6.0 Hz, 1H), 3.78 (s, 3H), 3.23 (dd, half of ABX pattern, J=15.7, 6.0 Hz, 1H), 3.14 (br dd, half of ABX pattern, J=15.8, 9.4 Hz, 1H), 1.47 (s, 9H).

### Step 2. Synthesis of 7-(3-methoxyphenoxy)-4,5-dihydro[1,2,4]triazolo[4,3-a]quinolin-4-amine, hydrochloride salt (C27)

tert-Butyl [7-(3-Methoxyphenoxy)-4,5-dihydro[1,2,4]triazolo[4,3-a]quinolin-4-yl]carbamate (C26) (225 mg, 0.551 mmol) was dissolved in a solution of hydrogen chloride in 2-propanol (5 M, 10 mL) and the reaction mixture was allowed to stir at room temperature for 2 hours. Removal of solvent in vacuo provided a paste, which was slurried in diethyl ether (100 mL). The solids were collected via filtration and washed with diethyl ether to afford the product as a pale yellow solid. Yield: 179 mg, 0.519 mmol, 94%. LCMS m/z 309.2 [M+H⁺]. ¹H NMR (400 MHz, CD₃OD) δ 9.58 (s, 1H), 7.80 (d, J=8.8 Hz, 1H), 7.28-7.33 (m, 1H), 7.17 (br d, J=2.5 Hz, 1H), 7.13 (br dd, J=8.7, 2.6 Hz, 1H), 6.78 (ddd, J=8.4, 2.2, 0.9 Hz, 1H), 6.61-6.64 (m, 2H), 5.10 (dd, J=9.7, 6.1 Hz, 1H), 3.79 (s, 3H), 3.52 (dd, J=16.1, 6.1 Hz, 1H), 3.23-3.31 (m, 1H, assumed; partially obscured by solvent peak).

### Step 3. Isolation of 7-(3-methoxyphenoxy)-4,5-dihydro[1,2,4]triazolo[4,3-a]quinolin-4-amine, ENT-1 (4) and 7-(3-methoxyphenoxy)-4,5-dihydro[1,2,4]triazolo[4,3-a]quinolin-4-amine, ENT-2 (5)

A sample of 7-(3-methoxyphenoxy)-4,5-dihydro[1,2,4]triazolo[4,3-a]quinolin-4-amine, hydrochloride salt (C27) was subjected to supercritical fluid chromatography (Column: Chiral Technologies Chiralcel AS-H, 5 μm; Eluent: 65:35 carbon dioxide/methanol containing 0.2% isopropylamine). The first-eluting enantiomer was 7-(3-methoxyphenoxy)-4,5-dihydro[1,2,4]triazolo[4,3-a]quinolin-4-amine ENT-1 (4), obtained as a solid. Retention time: 4.39 minutes (Column: Chiral Technologies Chiralcel AS-H, 5 μm, 4.6×25 mm; Eluent: 65:35 carbon dioxide/methanol containing 0.2% isopropylamine; Flow rate 2.5 ml/min). LCMS m/z 309.1 [M+H⁺]. Enantiomer 7-(3-methoxyphenoxy)-4,5-dihydro[1,2,4]triazolo[4,3-a]quinolin-4-amine ENT-2 (5), the second-eluting compound, was also collected as a solid; retention time: 5.19 minutes under the same conditions. LCMS m/z 309.1 [M+H⁺].

### Example 6

### (4S)-4-Amino-7-(3-methoxyphenoxy)-4,5-dihydro[1,2,4]oxadiazolo[4,3-a]quinolin-1-one, hydrochloride salt (6)

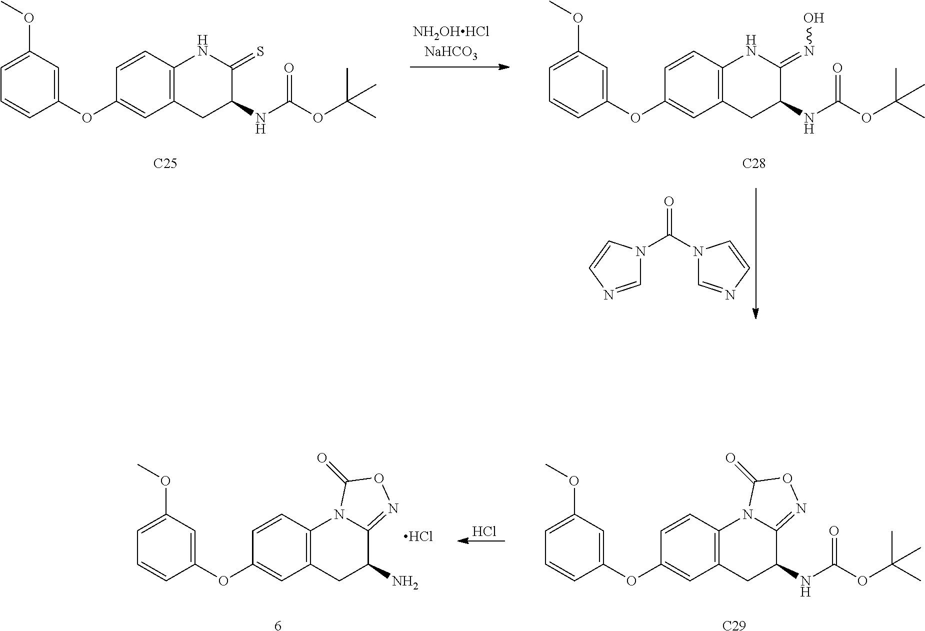

### Step 1. Synthesis of tert-butyl [(3S)-2-(hydroxyimino)-6-(3-methoxyphenoxy)-1,2,3,4-tetrahydroquinolin-3-yl]carbamate (C28)

To a solution of tert-butyl [(3S)-6-(3-methoxyphenoxy)-2-thioxo-1,2,3,4-tetrahydroquinolin-3-yl]carbamate (C25) (1.0 g, 2.5 mmol) in methanol (20 mL) was added hydroxylamine hydrochloride (0.34 g, 4.9 mmol) followed by sodium bicarbonate (0.52 g, 6.2 mmol). The reaction mixture was heated to reflux for 3 hours, then concentrated in vacuo and partitioned between ethyl acetate and water. The organic layer was dried over sodium sulfate, filtered and concentrated under reduced pressure; the residue was triturated with diethyl ether and pentane to afford the product as a brown solid. Yield: 800 mg, 2.00 mmol, 80%. LCMS m/z 400.1 [M+H⁺].

### Step 2. Synthesis of tert-butyl [(4S)-7-(3-methoxyphenoxy)-1-oxo-4,5-dihydro[1,2,4]oxadiazolo[4,3-a]quinolin-4-yl]carbamate (C29)

1,1′-Carbonyldiimidazole (1.2 g, 7.4 mmol) was added to a solution of tert-butyl [(3S)-2-(hydroxyimino)-6-(3-methoxyphenoxy)-1,2,3,4-tetrahydroquinolin-3-yl]carbamate (C28) (0.60 g, 1.5 mmol) in dichloromethane (30 mL) and the reaction mixture was stirred at room temperature for 3 hours, then partitioned between ethyl acetate and water. The organic layer was dried over sodium sulfate, filtered, and concentrated in vacuo. Purification was carried out via chromatography on silica gel (Eluent: 5% ethyl acetate in petroleum ether) to afford the product as an off-white solid. Yield: 360 mg, 0.85 mmol, 57%. ¹H NMR (400 MHz, DMSO-d₆) δ 7.99 (d, J=8.8 Hz, 1H), 7.65 (br d, J=8 Hz, 1H), 7.29 (dd, J=8.4, 8.1 Hz, 1H), 7.14 (br d, J=2.6 Hz, 1H), 7.08 (br dd, J=8.8, 2.8 Hz, 1H), 6.74 (ddd, J=8.3, 2.4, 0.8 Hz, 1H), 6.60 (dd, J=2.3, 2.3 Hz, 1H), 6.57 (ddd, J=8.0, 2.3, 0.8 Hz, 1H), 4.95-5.05 (m, 1H), 3.74 (s, 3H), 3.18 (dd, half of ABX pattern, J=15.7, 5.9 Hz, 1H), 3.09 (dd, half of ABX pattern, J=15.7, 10.4 Hz, 1H), 1.41 (s, 9H).

### Step 3. Synthesis of (4S)-4-amino-7-(3-methoxyphenoxy)-4,5-dihydro[1,2,4]oxadiazolo[4,3-a]quinolin-1-one, hydrochloride salt (6)

tert-Butyl [(4S)-7-(3-methoxyphenoxy)-1-oxo-4,5-dihydro[1,2,4]oxadiazolo[4,3-a]quinolin-4-yl]carbamate (C29) (0.36 g, 0.85 mmol) was stirred with a solution of hydrogen chloride in diethyl ether (4 M, 10 mL) at room temperature for 2 hours. After removal of solvent in vacuo, the residue was triturated with diethyl ether and pentane under argon to afford the product as a white solid. Yield: 210 mg, 0.58 mmol, 68%. LCMS m/z 326.3 [M+H⁺]. ¹H NMR (300 MHz, DMSO-d₆) δ 9.08 (br s, 3H), 8.01 (d, J=9.1 Hz, 1H), 7.31 (dd, J=8.4, 7.7 Hz, 1H), 7.20-7.25 (m, 1H), 7.15 (br dd, J=9, 2 Hz, 1H), 6.76 (br d, J=8.4 Hz, 1H), 6.54-6.63 (m, 2H), 4.96 (dd, J=9.8, 6.3 Hz, 1H), 3.75 (s, 3H), 3.19-3.47 (m, 2H, assumed; partially obscured by water peak).

### Example 7

### 7-Phenoxy-4,5-dihydro[1,2,4]triazolo[4,3-a][1,7]naphthyridin-4-amine, hydrochloride salt (7)

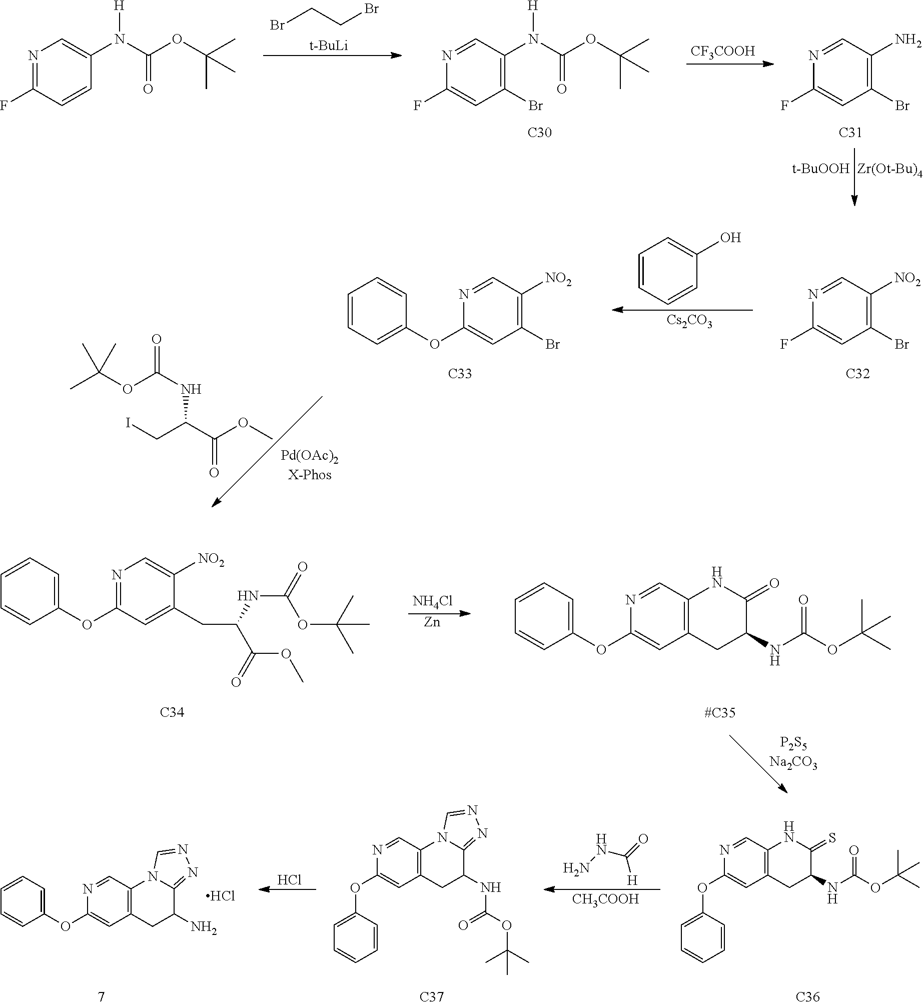

### Step 1. Synthesis of tert-butyl (4-bromo-6-fluoropyridin-3-yl)carbamate (C30)

A solution of tert-butyl (6-fluoropyridin-3-yl)carbamate (see A. Wissner et al., Bioorg. Med. Chem. Lett. 2004, 14, 1411-1416) (50 g, 240 mmol) in tetrahydrofuran (1 L) was cooled to −78° C. and treated with tert-butyllithium (1.5 M solution in pentane, 628 mL, 942 mmol) in a drop-wise addition. The reaction mixture was stirred for 1 hour at −40° C. and re-cooled to −78° C. A solution of 1,2-dibromoethane (61 mL, 710 mmol) in tetrahydrofuran was added drop-wise, and the reaction mixture was stirred at −78° C. for 2 hours and at room temperature for 12 hours. After dilution with aqueous ammonium chloride solution, the mixture was extracted with ethyl acetate. The combined organic layers were dried over sodium sulfate, filtered, and concentrated in vacuo. Chromatography on silica gel (Eluent: 2% ethyl acetate in petroleum ether) provided the product as a yellow solid. Yield: 20 g, 69 mmol, 29%. LCMS m/z 235.0, 237.0 {[M−(2-methylprop-1-ene)]+H⁺}. ¹H NMR (300 MHz, DMSO-d₆) δ 8.96 (br s, 1H), 8.22 (s, 1H), 7.68 (d, J=3.1 Hz, 1H), 1.45 (s, 9H).

### Step 2. Synthesis of 4-bromo-6-fluoropyridin-3-amine (C31)

To a 0° C. solution of tert-butyl (4-bromo-6-fluoropyridin-3-yl)carbamate (C30) (20 g, 69 mmol) in dichloromethane (150 mL) was added trifluoroacetic acid (150 mL) and the reaction mixture was stirred for 2 hours at room temperature. Additional dichloromethane was added and the mixture was washed with aqueous sodium bicarbonate solution. The organic layer was dried over sodium sulfate, filtered and concentrated under reduced pressure; recrystallization from 1:1 n-hexane/dichloromethane afforded the product as a brown solid. Yield: 10 g, 52 mmol, 75%. LCMS m/z 191.0, 193.0 [M+H⁺]. ¹H NMR (300 MHz, DMSO-d₆) δ 7.65 (d, J=1.7 Hz, 1H), 7.33 (d, J=3.5 Hz, 1H), 5.45 (br s, 2H).

### Step 3. Synthesis of 4-bromo-2-fluoro-5-nitropyridine (C32)

To a 0° C. solution of 4-bromo-6-fluoropyridin-3-amine (C31) (6.0 g, 31 mmol) in dichloromethane (120 mL) was added 4 angstrom molecular sieves (6 g), followed by zirconium(IV) tert-butoxide (6.03 g, 15.7 mmol), and tert-butyl hydroperoxide (5.5 M solution in n-decane, 28.6 mL, 157 mmol). The reaction mixture was stirred for 4 hours, and then quenched with 5% aqueous hydrochloric acid. The mixture was passed through a pad of Celite, and the organic layer from the filtrate was dried over sodium sulfate, filtered, and concentrated in vacuo. Chromatography on silica gel (Eluent: 3% ethyl acetate in petroleum ether) provided the product as an off-white solid. Yield: 2.35 g, 10.6 mmol, 34%. GCMS m/z 220.0 [M⁺]. ¹H NMR (300 MHz, CDCl₃) δ 8.85 (s, 1H), 7.40 (d, J=2.8 Hz, 1H).

### Step 4. Synthesis of 4-bromo-5-nitro-2-phenoxypyridine (C33)

To a 0° C. solution of 4-bromo-2-fluoro-5-nitropyridine (C32) (0.60 g, 2.7 mmol) in acetonitrile (25 mL) was added cesium carbonate (0.97 g, 3.0 mmol) followed by phenol (0.28 g, 3.0 mmol) and the reaction mixture was stirred for 2 hours at 0° C. The reaction mixture was then diluted with water and extracted with ethyl acetate; the combined organic layers were dried over sodium sulfate, filtered, and concentrated in vacuo. Purification was carried out by chromatography on silica gel (Eluent: 3% ethyl acetate in petroleum ether), affording the product as an off-white solid. Yield: 0.60 g, 2.0 mmol, 74%. LCMS m/z 295.1, 297.1 [M+H⁺]. ¹H NMR (300 MHz, CDCl₃) δ 8.80 (s, 1H), 7.43-7.52 (m, 2H), 7.30 (s, 1H), 7.29-7.36 (m, 1H), 7.13-7.19 (m, 2H).

### Step 5. Synthesis of methyl (2S)-[2-(tert-butoxycarbonylamino)]-3-(5-nitro-2-phenoxypyridin-4-yl)propanoate (C34)

4-Bromo-5-nitro-2-phenoxypyridine (C33) was converted to the product using the method described for synthesis of tert-butyl [(3R)-6-(3-methoxyphenoxy)-2-oxo-1,2,3,4-tetrahydroquinolin-3-yl]carbamate (C3) in Example 1, except that methyl N-(tert-butoxycarbonyl)-3-iodo-L-alaninate was used in place of methyl N-(tert-butoxycarbonyl)-3-iodo-D-alaninate. The product was obtained as a yellow oil. Yield: 211 mg, 0.505 mmol, 60%. LCMS m/z 418.3 [M+H⁺]. ¹H NMR (400 MHz, CDCl₃) δ 8.88 (br s, 1H), 7.45 (br dd, J=8.4, 7.6 Hz, 2H), 7.29 (br t, J=7.4 Hz, 1H), 7.11-7.16 (m, 2H), 6.89 (br s, 1H), 5.24 (br d, J=8.0 Hz, 1H), 4.69-4.80 (m, 1H), 3.78 (s, 3H), 3.69 (dd, J=13.4, 5.2 Hz, 1H), 3.23 (br dd, J=13, 9 Hz, 1H), 1.39 (s, 9H).

### Step 6. Synthesis of tert-butyl [(3S)-2-oxo-6-phenoxy-1,2,3,4-tetrahydro-1,7-naphthyridin-3-yl]carbamate (C35)

Methyl (2S)-[2-(tert-butoxycarbonylamino)]-3-(5-nitro-2-phenoxypyridin-4-yl)propanoate (C34) was converted to the product using the method described for synthesis of tert-butyl [(3S)-2-oxo-6-phenoxy-1,2,3,4-tetrahydro-1,8-naphthyridin-3-yl]carbamate (C11) in Example 2. The product was obtained as a white foam. Yield: 104 mg, 0.293 mmol, 58%. ¹H NMR (400 MHz, CDCl₃) δ 7.74 (s, 1H), 7.62 (br s, 1H), 7.41 (br dd, J=8.5, 7.5 Hz, 2H), 7.19-7.24 (m, 1H), 7.09-7.13 (m, 2H), 6.78 (br s, 1H), 5.63 (v br s, 1H), 4.29-4.38 (m, 1H), 3.55 (br dd, J=15, 5 Hz, 1H), 2.84 (br dd, J=15, 14 Hz, 1H), 1.48 (s, 9H).

### Step 7. Synthesis of tert-butyl [(3S)-6-phenoxy-2-thioxo-1,2,3,4-tetrahydro-1,7-naphthyridin-3-yl]carbamate (C36)

tert-Butyl [(3S)-2-oxo-6-phenoxy-1,2,3,4-tetrahydro-1,7-naphthyridin-3-yl]carbamate (C35) was converted to the product according to the method described for synthesis of tert-butyl [(3S)-6-phenoxy-2-thioxo-1,2,3,4-tetrahydro-1,8-naphthyridin-3-yl]carbamate (C12) in Example 2. The product was obtained as a yellow foam. Yield: 76 mg, 0.20 mmol, 68%. LCMS m/z 372.1 [M+H⁺]. ¹H NMR (400 MHz, CDCl₃) δ 9.89 (br s, 1H), 7.85 (s, 1H), 7.41 (br dd, J=8.5, 7.5 Hz, 2H), 7.20-7.25 (m, 1H), 7.10-7.14 (m, 2H), 6.77 (br s, 1H), 6.09-6.16 (br m, 1H), 4.32-4.40 (m, 1H), 3.45-3.57 (br m, 1H), 2.72 (br dd, J=15, 15 Hz, 1H), 1.50 (s, 9H).

### Step 8. Synthesis of tert-butyl [7-phenoxy-4,5-dihydro[1,2,4]triazolo[4,3-a][1,7]naphthyridin-4-yl]carbamate (C37)

tert-Butyl [(3S)-6-phenoxy-2-thioxo-1,2,3,4-tetrahydro-1,7-naphthyridin-3-yl]carbamate (C36) was converted to the product using the method described for the synthesis of tert-butyl [7-(3-methoxyphenoxy)-4,5-dihydro[1,2,4]triazolo[4,3-a]quinolin-4-yl]carbamate (C26) in Example 4/Example 5. The product was obtained as an orange foam. Yield: 43 mg, 0.11 mmol, 58%. LCMS m/z 380.4 [M+H⁺]. ¹H NMR (400 MHz, CDCl₃) δ 8.65 (s, 1H), 8.31 (s, 1H), 7.44 (br dd, J=8.5, 7.5 Hz, 2H), 7.23-7.29 (m, 1H), 7.12-7.16 (m, 2H), 6.94 (br s, 1H), 5.66-5.76 (br m, 1H), 5.18 (ddd, J=11.1, 6.1, 5.8 Hz, 1H), 3.48-3.61 (br m, 1H), 2.98 (dd, J=15.6, 11.3 Hz, 1H), 1.49 (s, 9H).

### Step 9. Synthesis of 7-phenoxy-4,5-dihydro[1,2,4]triazolo[4,3-a][1,7]naphthyridin-4-amine, hydrochloride salt (7)

tert-Butyl [7-phenoxy-4,5-dihydro[1,2,4]triazolo[4,3-a][1,7]naphthyridin-4-yl]carbamate (C37) was converted to the product according to the method used for synthesis of 6-amino-3-phenoxy-6,8-dihydro[1,2,4]triazolo[4,3-a][1,8]naphthyridin-9(5H)-one, hydrochloride salt (C14) in Example 2. In this case, after chromatography, formation of the hydrochloride salt was carried out using a solution of hydrogen chloride in 2-propanol (5-6 M). The product was obtained as an off-white solid. Yield: 31 mg, 0.098 mmol, 98%. LCMS m/z 280.3 [M+H⁺]. ¹H NMR (400 MHz, CD₃OD) δ 9.53 (s, 1H), 8.60 (s, 1H), 7.44 (br dd, J=7.6, 7.6 Hz, 2H), 7.26 (br dd, J=7.4, 7.4 Hz, 1H), 7.13-7.18 (m, 3H), 5.14 (br dd, J=9.7, 6.2 Hz, 1H), 3.62 (br dd, J=16.5, 6.3 Hz, 1H), 3.3-3.37 (m, 1H, assumed; partially obscured by solvent peak). {Neutral form of 7: ¹H NMR (400 MHz, CD₃OD) δ 9.23 (s, 1H), 8.52 (s, 1H), 7.43 (br dd, J=8.5, 7.5 Hz, 2H), 7.21-7.26 (m, 1H), 7.11-7.16 (m, 2H), 7.07 (br s, 1H), 4.56 (dd, J=8.0, 5.5 Hz, 1H), 3.32 (ddd, J=16.4, 5.7, 0.8 Hz, 1H), 3.10 (ddd, J=16.4, 8.0, 0.8 Hz, 1H).}

### Preparation P1

### tert-Butyl (7-bromo-1-oxo-1,2,4,5-tetrahydro[1,2,4]triazolo[4,3-a]quinolin-4-yl)carbamate (P1)

Preparation P1 describes preparations of certain intermediates that can be used for preparation of certain compounds of the invention

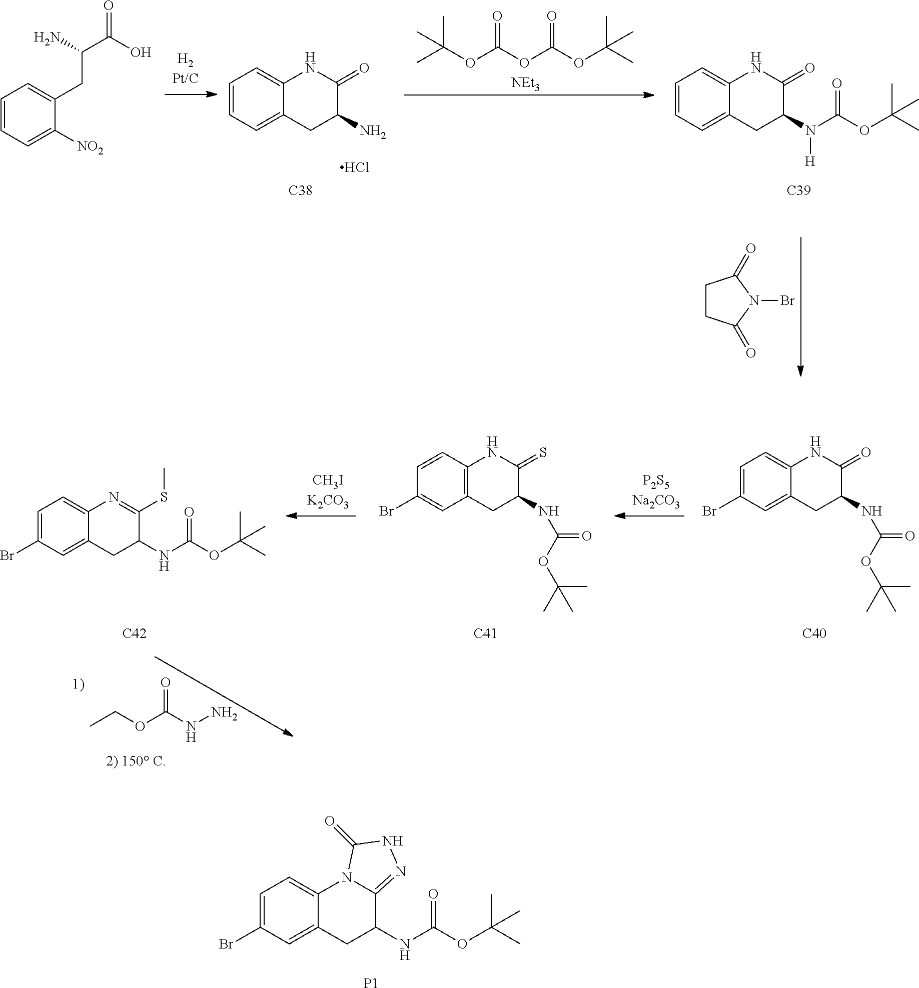

### Step 1. Synthesis of (3S)-3-amino-3,4-dihydroquinolin-2(1H)-one, hydrochloride salt (C38)

Water (5 mL) and concentrated hydrochloric acid (15 mL) were added to a suspension of 2-nitro-L-phenylalanine (10 g, 48 mmol) in methanol (470 mL). Platinum on activated carbon (5% by weight, 3 g) was added to the resulting solution, and the reaction mixture was hydrogenated in a Parr shaker at 60 psi for 3 hours. The reaction mixture was filtered though a pad of Celite and concentrated in vacuo; trituration with hexanes/diethyl ether afforded the product as a brown solid. Yield: 9.0 g, 45 mmol, 94%. LCMS m/z 163.2 [M+H⁺].

### Step 2. Synthesis of tert-butyl [(3S)-2-oxo-1,2,3,4-tetrahydroquinolin-3-yl]carbamate (C39)

To a 0° C. solution of (3S)-3-amino-3,4-dihydroquinolin-2(1H)-one, hydrochloride salt (C38) (30 g, 150 mmol) in 1,4-dioxane/water (1:1, 600 mL) was added triethylamine (128 mL, 918 mmol) followed by di-tert-butyl dicarbonate (52.2 mL, 227 mmol), and the reaction mixture was stirred at room temperature for 2 hours. Most of the 1,4-dioxane was removed under reduced pressure while maintaining the bath temperature below 40° C. The aqueous residue was extracted with ethyl acetate; the combined organic layers were washed with water and with saturated aqueous sodium chloride solution, dried over sodium sulfate, and concentrated in vacuo. Purification via chromatography on silica gel (Eluent: 20% ethyl acetate in petroleum ether) provided the product as an off-white solid. Yield: 11 g, 42 mmol, 28%. LCMS m/z 207.2 {[M−(2-methylprop-1-ene)]+H⁺}. ¹H NMR (300 MHz, DMSO-d₆) δ 10.21 (s, 1H), 7.12-7.21 (m, 2H), 6.82-7.00 (m, 3H), 4.07-4.23 (m, 1H), 2.89-3.01 (m, 2H), 1.41 (s, 9H).

### Step 3. Synthesis of tert-butyl [(3S)-6-bromo-2-oxo-1,2,3,4-tetrahydroquinolin-3-yl]carbamate (C40)

To a solution of tert-butyl [(3S)-2-oxo-1,2,3,4-tetrahydroquinolin-3-yl]carbamate (C39) (13 g, 50 mmol) in N,N-dimethylformamide (100 mL) was added a solution of N-bromosuccinimide (10.6 g, 59.6 mmol) in N,N-dimethylformamide (56 mL). The reaction mixture was stirred at room temperature for 8 hours, then poured into ice water and extracted with ethyl acetate. The combined organic layers were washed with water and with saturated aqueous sodium chloride solution, dried over sodium sulfate, filtered, and concentrated under reduced pressure. Purification via silica gel chromatography (Eluent: 20% ethyl acetate in petroleum ether) afforded the product as a pale yellow solid. Yield: 10.6 g, 31.1 mmol, 62%. LCMS m/z 285.1 {[M−(2-methylprop-1-ene)]+H⁺}. ¹H NMR (300 MHz, DMSO-d₆) δ 10.31 (s, 1H), 7.41 (br d, J=2 Hz, 1H), 7.34 (br dd, J=8.4, 2.1 Hz, 1H), 7.00 (br d, J=8.7 Hz, 1H), 6.80 (d, J=8.4 Hz, 1H), 4.08-4.23 (m, 1H), 2.86-3.06 (m, 2H), 1.40 (s, 9H).

### Step 4. Synthesis of tert-butyl [(3S)-6-bromo-2-thioxo-1,2,3,4-tetrahydroquinolin-3-yl]carbamate (C41)

tert-Butyl [(3S)-6-bromo-2-oxo-1,2,3,4-tetrahydroquinolin-3-yl]carbamate (C40) was converted to the product according to the general procedure for the synthesis of tert-butyl [(3R)-6-(3-methoxyphenoxy)-2-thioxo-1,2,3,4-tetrahydroquinolin-3-yl]carbamate (C4) in Example 1. The product was obtained as a yellow solid. Yield: 8.8 g, 25 mmol, 78%. LCMS m/z 356.7, 358.7 [M+H⁺]. ¹H NMR (400 MHz, DMSO-d₆) δ 12.36 (s, 1H), 7.48 (br d, J=2 Hz, 1H), 7.42 (br dd, J=8.5, 2.2 Hz, 1H), 7.09 (br d, J=7.9 Hz, 1H), 7.03 (d, J=8.4 Hz, 1H), 4.22-4.33 (m, 1H), 3.03 (dd, J=16.0, 6.0 Hz, 1H), 2.82 (dd, J=15.8, 13.3 Hz, 1H), 1.41 (s, 9H).

### Step 5. Synthesis of tert-butyl [6-bromo-2-(methylsulfanyl)-3,4-dihydroquinolin-3-yl]carbamate (C42)

The product was prepared from tert-butyl [(3S)-6-bromo-2-thioxo-1,2,3,4-tetrahydroquinolin-3-yl]carbamate (C41) according to the general procedure for the synthesis of tert-butyl [6-(3-methoxyphenoxy)-2-(methylsulfanyl)-3,4-dihydroquinolin-3-yl]carbamate (C5) in Example 1. In this case, the reaction mixture was filtered to remove solids; the solids were washed with ethyl acetate, and the combined filtrates were concentrated in vacuo to provide the product (5.5 g). By ¹H NMR analysis, this contained residual tetrahydrofuran and ethyl acetate. Corrected yield: 5.1 g, 13.7 mmol, 98%. LCMS m/z 371.0, 373.0 [M+H⁺]. ¹H NMR (400 MHz, CD₃OD) δ 7.37 (dd, J=8.3, 2.3 Hz, 1H), 7.29-7.32 (m, 1H), 7.14 (d, J=8.4 Hz, 1H), 4.38 (dd, J=10.8, 9.5 Hz, 1H), 2.81-2.91 (m, 2H), 2.42 (s, 3H), 1.47 (s, 9H).

### Step 6. Synthesis of tert-butyl (7-bromo-1-oxo-1,2,4,5-tetrahydro[1,2,4]triazolo[4,3-a]quinolin-4-yl)carbamate (P1)

The product was prepared from tert-butyl [6-bromo-2-(methylsulfanyl)-3,4-dihydroquinolin-3-yl]carbamate (C42) according to the general procedure for the conversion of tert-butyl [6-(3-methoxyphenoxy)-2-(methylsulfanyl)-3,4-dihydroquinolin-3-yl]carbamate (C5) to racemic tert-butyl [7-(3-methoxyphenoxy)-1-oxo-1,2,4,5-tetrahydro[1,2,4]triazolo[4,3-a]quinolin-4-yl]carbamate (C7/C8) in Example 1. In this case, the crude hydrazinecarboxylate intermediate was taken directly into the thermal cyclization. The final organic extracts were washed with water and with saturated aqueous sodium chloride solution, dried over magnesium sulfate, filtered, and concentrated in vacuo. The resulting foam was suspended in dichloromethane (100 mL) and concentrated under reduced pressure to yield a solid, which was suspended in a mixture of heptane and dichloromethane (10:1, 150 mL). The solid was then collected by filtration and washed with heptane to afford the product as a tan solid. Yield: 4.06 g, 10.6 mmol, 77%. LCMS m/z 379.0, 381.1 [M−H⁺]. ¹H NMR (400 MHz, CD₃OD) δ 8.18 (d, J=8.4 Hz, 1H), 7.50-7.55 (m, 2H), 4.90 (dd, J=10.1, 5.6 Hz, 1H), 3.18 (dd, half of ABX pattern, J=15.6, 5.7 Hz, 1H), 3.06 (dd, half of ABX pattern, J=15.5, 10.1 Hz, 1H), 1.47 (s, 9H).

### Method A

### Synthesis of 7-substituted 4-amino-4,5-dihydro[1,2,4]triazolo[4,3-a]quinolin-1(2H)-ones via Suzuki reaction

Method A describes a specific method for preparation of certain compounds of the invention.

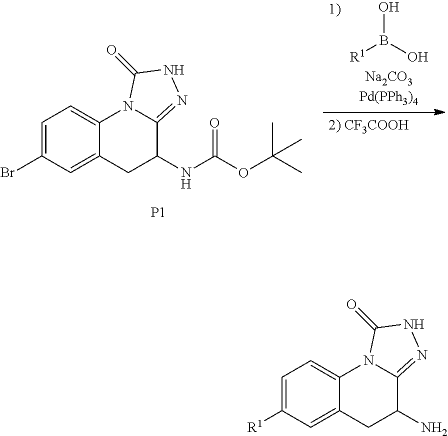

Ethanol and toluene solvents were degassed for 1 hour with a stream of nitrogen. A fine suspension of tert-butyl (7-bromo-1-oxo-1,2,4,5-tetrahydro[1,2,4]triazolo[4,3-a]quinolin-4-yl)carbamate (P1) (23 mg, 0.06 mmol) in ethanol (0.5 mL) was added to the appropriate boronic acid (0.078 mmol). A solution of sodium carbonate (38 mg, 0.36 mmol) in water (0.1 mL) was added, followed by a solution of tetrakis(triphenylphosphine)palladium(0) (4.2 mg, 0.0036 mmol) in toluene (0.5 mL). The reaction mixture was degassed via two rounds of vacuum evacuation followed by nitrogen fill, then shaken and heated at 95° C. for 20 hours. After cooling, the reaction mixture was partitioned between aqueous sodium hydroxide solution (1 M, 1.5 mL) and ethyl acetate (2.5 mL) and vortexed. The organic layer was passed through a solid phase extraction cartridge containing sodium sulfate (6 mL cartridge, approximately 1 g bed weight). This extraction was repeated twice and the combined extracts were concentrated in vacuo. The residue was treated with a mixture of trifluoroacetic acid and 1,2-dichloroethane (1:1, 0.5 mL), and shaken at room temperature for 3 hours. After removal of solvent under reduced pressure, the residue was dissolved in a mixture of methanol and 1,2-dichloroethane (1:1, 2.5 mL), using heat and vortexing if necessary. The solution was loaded onto an SCX (strong cation exchanger) solid-phase extraction cartridge (Silicycle, 6 mL, 1 g bed weight), and the cartridge was rinsed twice with a mixture of methanol and 1,2-dichloroethane (1:1, 2.5 mL), followed by methanol (5 mL). The crude product was then eluted with a solution of triethylamine in methanol (1 M, 7.2 mL). After concentration in vacuo, dissolution in dimethyl sulfoxide (1 mL) and filtration through a Waters Oasis filter cartridge to remove particulates, purification was carried out via reversed-phase HPLC (Column: Waters Sunfire C18, 19×100 mm, 5 μm; Mobile phase A: 0.05% trifluoroacetic acid in water (v/v); Mobile phase B: 0.05% trifluoroacetic acid in acetonitrile (v/v); Gradient: 5.0% to 100% B).

Making non-critical changes, the following compounds in Table 1 were prepared using methods and preparations same as or similar to those discussed herein.

TABLE 1

| | | Method of | |
|---|---|---|---|
| | | Preparation; | ¹H NMR (400 MHz, CD₃OD), δ (ppm); Mass |
| | | Non- | spectrum, observed ion m/z (M + H⁺) or |
| | | commercial | HPLC retention time (minutes); Mass |
| Example | | Starting | spectrum m/z (M + H⁺) (unless otherwise |
| Number | Structure | Materials | indicated) |
| 8 | 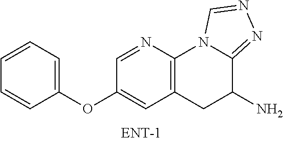 [SMILES: NC1Cc2cc(Oc3ccccc3)cnc2-n2cnnc21] | Ex 4; C12^{1,2} | 6.23 minutes³; 280.1 |
| 9 | 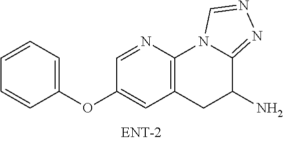 [SMILES: NC1Cc2cc(Oc3ccccc3)cnc2-n2cnnc21] | Ex 4; C12^{1,2} | 7.11 minutes³; 280.1 |
| 10 | 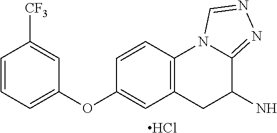 [SMILES: NC1Cc2cc(Oc3cccc(C(F)(F)F)c3)ccc2-n2cnnc21 ; Cl] | Ex 2, Ex 4¹⁰ | 9.87 (s, 1H), 7.89 (d, J = 8.8 Hz, 1H), 7.62 (br dd, J = 8, 8 Hz, 1H), 7.51 (br d, J = 8 Hz, 1H), 7.31-7.36 (m, 2H), 7.28 (br d, J = 2.3 Hz, 1H), 7.22 (br dd, J = 8.7, 2.8 Hz, 1H), 5.16 (dd, J = 10.0, 6.1 Hz, 1H), 3.58 (dd, J = 16.0, 6.2 Hz, 1H), 3.31-3.38 (dd, J = 16.0, 10.0 Hz, 1H, assumed; partially obscured by solvent peak); 347.0 |
| 11 | 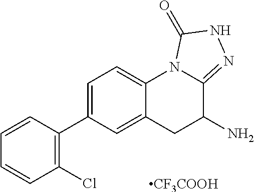 [SMILES: NC1Cc2cc(-c3ccccc3Cl)ccc2-n2c1n[nH]c2=O ; "·CF3COOH"] | Method A | 2.08 minutes⁴; 313.1, 315.1 |
| 12 | 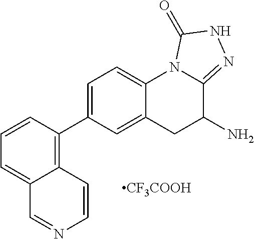 [SMILES: NC1Cc2cc(-c3cccc4cnccc34)ccc2-n2c1n[nH]c2=O ; "·CF3COOH"] | Method A | 1.29 minutes⁴; 330.1 |
| 13 | 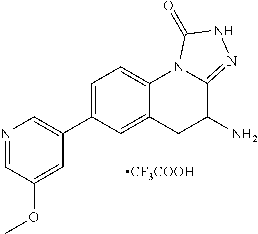 [SMILES: COc1cncc(-c2ccc3c(c2)CC(N)c2n[nH]c(=O)n2-3)c1 ; "·CF3COOH"] | Method A | 1.20 minutes⁴; 310.1 |
| 14 | 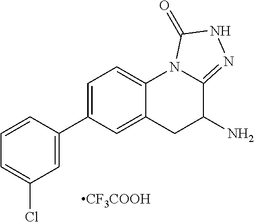 | Method A | 2.18 minutes⁴; 313.1, 315.1 |
| 15 | 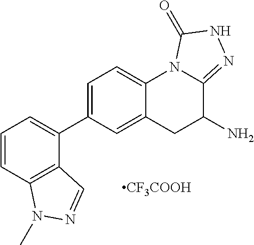 [SMILES: Cn1ncc2c(-c3ccc4c(c3)CC(N)c3n[nH]c(=O)n3-4)cccc21 ; "·CF3COOH"] | Method A | 1.88 minutes⁴; 333.1 |
| 16 | 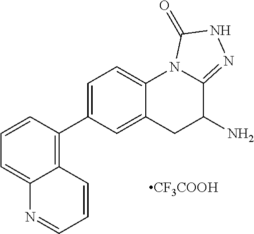 [SMILES: NC1Cc2cc(-c3cccc4ncccc34)ccc2-n2c1n[nH]c2=O ; "·CF3COOH"] | Method A | 1.27 minutes⁴; 330.1 |
| 17 | 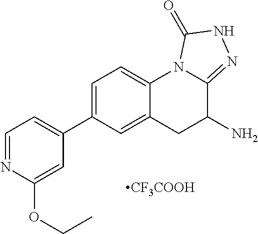 [SMILES: CCOc1cc(-c2ccc3c(c2)CC(N)c2n[nH]c(=O)n2-3)ccn1 ; "·CF3COOH"] | Method A | 1.53 minutes⁴; 324.1 |
| 18 | 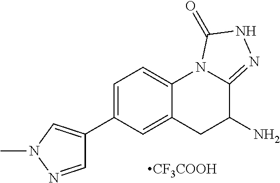 [SMILES: Cn1cc(-c2ccc3c(c2)CC(N)c2n[nH]c(=O)n2-3)cn1 ; "·CF3COOH"] | Method A | 1.52 minutes⁴; 283.0 |
| 19 | 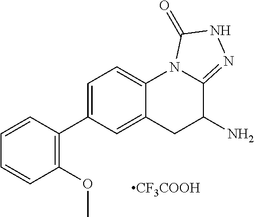 [SMILES: COc1ccccc1-c1ccc2c(c1)CC(N)c1n[nH]c(=O)n1-2 ; "·CF3COOH"] | Method A | 1.99 minutes⁴; 309.1 |
| 20 | 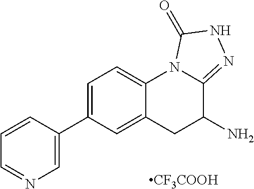 [SMILES: NC1Cc2cc(-c3cccnc3)ccc2-n2c1n[nH]c2=O ; "·CF3COOH"] | Method A | 0.99 minutes⁴; 280.1 |
| 21 | 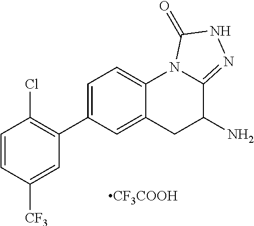 [SMILES: NC1Cc2cc(-c3cc(C(F)(F)F)ccc3Cl)ccc2-n2c1n[nH]c2=O ; "·CF3COOH"] | Method A | 2.37 minutes⁴; 381.0, 383.0 |
| 22 | 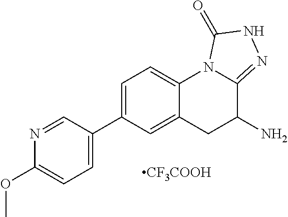 [SMILES: COc1ccc(-c2ccc3c(c2)CC(N)c2n[nH]c(=O)n2-3)cn1 ; "·CF3COOH"] | Method A | 1.63 minutes⁴; 310.1 |
| 23 | 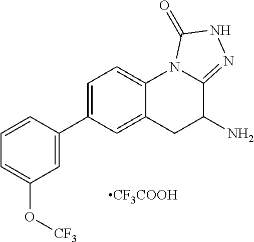 [SMILES: NC1Cc2cc(-c3cccc(OC(F)(F)F)c3)ccc2-n2c1n[nH]c2=O ; "·CF3COOH"] | Method A | 2.34 minutes⁴; 363.1 |
| 24 | 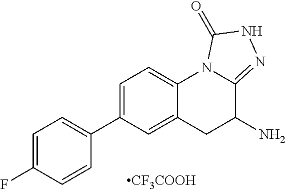 | Method A | 2.03 minutes⁴; 297.1 |
| 25 | 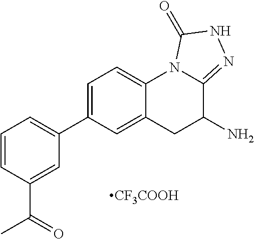 [SMILES: CC(=O)c1cccc(-c2ccc3c(c2)CC(N)c2n[nH]c(=O)n2-3)c1 ; "·CF3COOH"] | Method A | 1.85 minutes⁴; 321.1 |
| 26 | 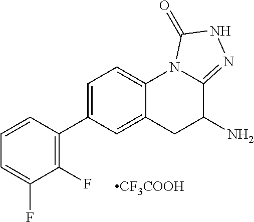 [SMILES: NC1Cc2cc(-c3cccc(F)c3F)ccc2-n2c1n[nH]c2=O ; "·CF3COOH"] | Method A | 2.06 minutes⁴; 315.1 |
| 27 | 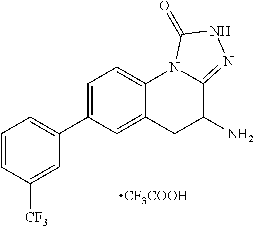 [SMILES: NC1Cc2cc(-c3cccc(C(F)(F)F)c3)ccc2-n2c1n[nH]c2=O ; "·CF3COOH"] | Method A | 2.28 minutes⁴; 347.1 |
| 28 | 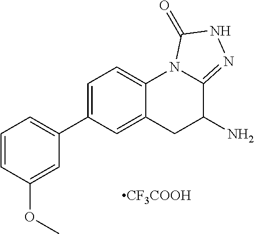 [SMILES: COc1cccc(-c2ccc3c(c2)CC(N)c2n[nH]c(=O)n2-3)c1 ; "·CF3COOH"] | Method A | 2.00 minutes⁴; 309.1 |
| 29 | 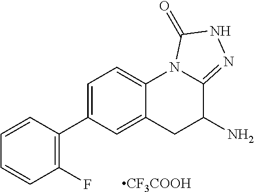 [SMILES: NC1Cc2cc(-c3ccccc3F)ccc2-n2c1n[nH]c2=O ; "·CF3COOH"] | Method A | 1.99 minutes⁴; 297.1 |
| 30 | 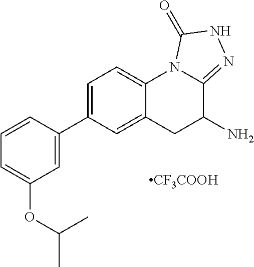 [SMILES: CC(C)Oc1cccc(-c2ccc3c(c2)CC(N)c2n[nH]c(=O)n2-3)c1 ; "·CF3COOH"] | Method A | 2.26 minutes⁴; 337.1 |
| 31 | 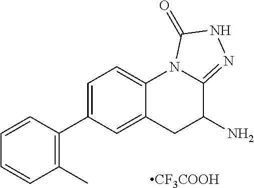 [SMILES: Cc1ccccc1-c1ccc2c(c1)CC(N)c1n[nH]c(=O)n1-2 ; "·CF3COOH"] | Method A | 2.09 minutes⁴; 293.1 |
| 32 | 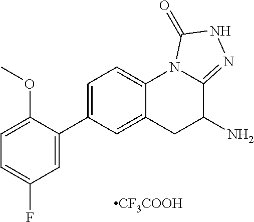 [SMILES: COc1ccc(F)cc1-c1ccc2c(c1)CC(N)c1n[nH]c(=O)n1-2 ; "·CF3COOH"] | Method A | 2.06 minutes⁴; 327.1 |
| 33 | 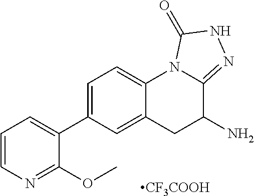 [SMILES: COc1ncccc1-c1ccc2c(c1)CC(N)c1n[nH]c(=O)n1-2 ; "·CF3COOH"] | Method A | 1.68 minutes⁴; 310.1 |
| 34 | 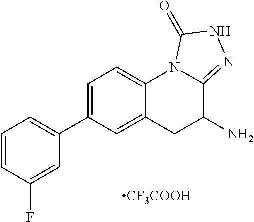 [SMILES: NC1Cc2cc(-c3cccc(F)c3)ccc2-n2c1n[nH]c2=O ; "·CF3COOH"] | Method A | 2.03 minutes⁴; 297.1 |
| 35 | 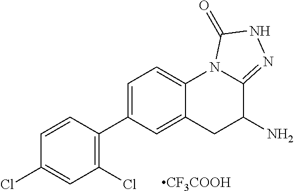 [SMILES: NC1Cc2cc(-c3ccc(Cl)cc3Cl)ccc2-n2c1n[nH]c2=O ; "·CF3COOH"] | Method A | 2.32 minutes⁴; 347.0, 349.0 |
| 36 | 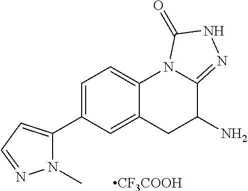 [SMILES: Cn1nccc1-c1ccc2c(c1)CC(N)c1n[nH]c(=O)n1-2 ; "·CF3COOH"] | Method A | 1.52 minutes⁴; 283.0 |
| 37 | 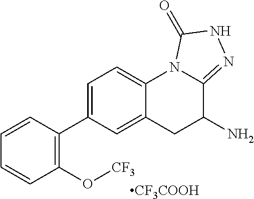 [SMILES: NC1Cc2cc(-c3ccccc3OC(F)(F)F)ccc2-n2c1n[nH]c2=O ; "·CF3COOH"] | Method A | 2.25 minutes⁴; 363.0 |
| 38 | 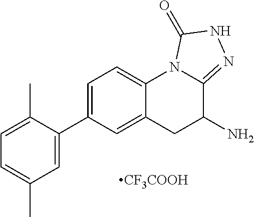 [SMILES: Cc1ccc(C)c(-c2ccc3c(c2)CC(N)c2n[nH]c(=O)n2-3)c1 ; "·CF3COOH"] | Method A | 2.23 minutes⁴; 307.1 |
| 39 | 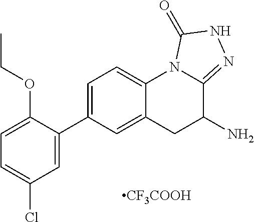 [SMILES: CCOc1ccc(Cl)cc1-c1ccc2c(c1)CC(N)c1n[nH]c(=O)n1-2 ; "·CF3COOH"] | Method A | 2.34 minutes⁴; 357.1, 359.1 |
| 40 | 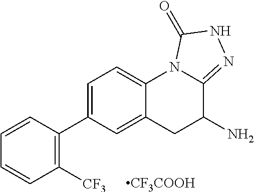 [SMILES: NC1Cc2cc(-c3ccccc3C(F)(F)F)ccc2-n2c1n[nH]c2=O ; "·CF3COOH"] | Method A | 2.18 minutes⁴; 347.0 |
| 41 | 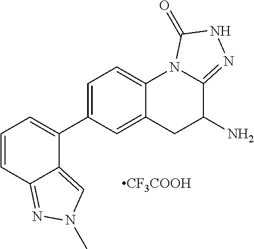 [SMILES: Cn1cc2c(-c3ccc4c(c3)CC(N)c3n[nH]c(=O)n3-4)cccc2n1 ; "·CF3COOH"] | Method A | 1.75 minutes⁴; 333.1 |
| 42 | 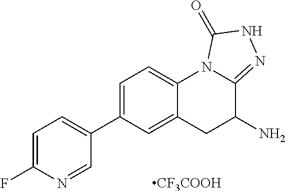 [SMILES: NC1Cc2cc(-c3ccc(F)nc3)ccc2-n2c1n[nH]c2=O ; "·CF3COOH"] | Method A | 1.62 minutes⁴; 298.1 |
| 43 | 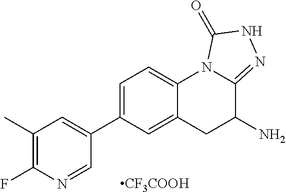 | Method A | 1.82 minutes⁴; 312.1 |
| 44 | 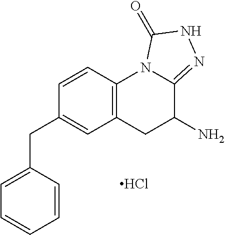 [SMILES: NC1Cc2cc(Cc3ccccc3)ccc2-n2c1n[nH]c2=O ; Cl] | Ex 2^{7,10} | 8.21 (d, J = 8.3 Hz, 1H), 7.16-7.34 (m, 7H), 4.72 (dd, J = 10.0, 5.8 Hz, 1H), 3.99 (s, 2H), 3.38 (br dd, J = 15.6, 5.8 Hz, 1H), 3.14 (br dd, J = 15.6, 10 Hz, 1H); 293 |
| 45 | 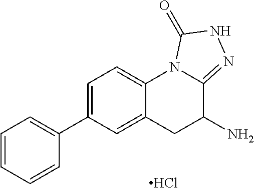 [SMILES: NC1Cc2cc(-c3ccccc3)ccc2-n2c1n[nH]c2=O ; Cl] | Method A⁸; P1 | 8.38 (d, J = 8.4 Hz, 1H), 7.69-7.74 (m, 2H), 7.63-7.67 (m, 2H), 7.46 (br dd, J = 8, 7 Hz, 2H), 7.35-7.40 (m, 1H), 4.81 (dd, J = 10.0, 5.8 Hz, 1H), 3.54 (dd, J = 15.6, 5.7 Hz, 1H), 3.24-3.3 (m, 1H, assumed; partially obscured by solvent peak); 279.1 |
| 46 | 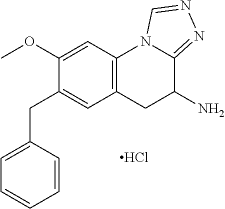 [SMILES: COc1cc2c(cc1Cc1ccccc1)CC(N)c1nncn1-2 ; Cl] | Ex 4^{11,10} | ¹H NMR (500 MHz, CD₃OD), δ 9.23 (s, 1H), 7.33 (s, 1H), 7.18-7.26 (m, 4H), 7.14 (s, 1H), 7.12-7.16 (m, 1H), 4.40 (dd, J = 8.8, 5.9 Hz, 1H), 3.94 (s, 2H), 3.91 (s, 3H), 3.13 (dd, J = 15.7, 5.7 Hz, 1H), 2.89 (dd, J = 15.7, 8.9 Hz, 1H)¹²; 307.1 |
| 47 | 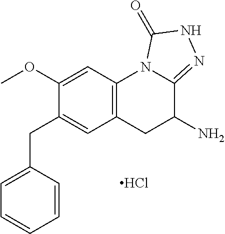 [SMILES: COc1cc2c(cc1Cc1ccccc1)CC(N)c1n[nH]c(=O)n1-2 ; Cl] | Ex 2^{11,10} | ¹H NMR (500 MHz, CD₃OD), δ 7.94 (s, 1H), 7.17-7.25 (m, 4H), 7.12-7.16 (m, 1H), 7.04 (s, 1H), 4.08 (dd, J = 9.8, 5.6 Hz, 1H), 3.93 (s, 2H), 3.86 (s, 3H), 3.05 (dd, J = 15.4, 5.4 Hz, 1H), 2.80 (dd, J = 15.4, 9.8 Hz, 1H)¹²; LCMS m/z 321.2 (M − H⁺) |
| 48 | 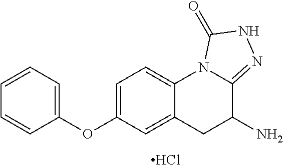 [SMILES: NC1Cc2cc(Oc3ccccc3)ccc2-n2c1n[nH]c2=O ; Cl] | Ex 3¹³ | ¹H NMR (300 MHz, DMSO-d₆), δ 12.32 (s, 1H), 8.85 (br s, 3H), 8.24 (d, J = 8.7 Hz, 1H), 7.37-7.45 (m, 2H), 7.13-7.20 (m, 2H), 7.08 (dd, J = 8.7, 2.8 Hz, 1H), 6.99-7.05 (m, 2H), 4.73-4.82 (m, 1H), 3.3-3.41 (m, 1H, assumed; partially obscured by water peak), 3.15 (dd, J = 16.2, 9.6 Hz, 1H); 295.0 |

1. Chiral separation was carried out via supercritical fluid chromatography (Column: Chiral Technologies Chiralpak AS-H, 5 μm; Eluent: 80:20 carbon dioxide/methanol containing 0.2% isopropylamine).
2. Example 8 was the first-eluting enantiomer from the column; Example 9 was the second-eluting enantiomer.
3. Supercritical fluid chromatography conditions. Column: Chiral Technologies Chiralpak AS-H, 5 μm, 4.6×25 mm; Eluent: 80:20 carbon dioxide/methanol containing 0.2% isopropylamine; Flow rate: 2.5 mL/minute.
4. Conditions for analytical HPLC. Column: Waters Atlantis dC18, 4.6×50 mm, 5 μm; Mobile phase A: 0.05% trifluoroacetic acid in water (v/v); Mobile phase B: 0.05% trifluoroacetic acid in acetonitrile (v/v); Gradient: 5.0% to 95% B, linear over 4.0 minutes; Flow rate: 2 mL/minute.
5. In this case, 2-bromo-4-fluoro-1-nitrobenzene was reacted with sodium methoxide to afford 2-bromo-4-methoxy-1-nitrobenzene.
6. Starting material 2-bromo-4-(trifluoromethoxy)aniline may be prepared according to J. Lau et al., J. Med. Chem. 2007, 50, 113-128.
7. 4-Benzylaniline was brominated using N-bromosuccinimide, to afford 4-benzyl-2-bromoaniline. This compound was converted to tert-butyl [(3S)-6-benzyl-2-oxo-1,2,3,4-tetrahydroquinolin-3-yl]carbamate using the chemistry described in Example 1; in this case, methyl N-(tert-butoxycarbonyl)-3-iodo-L-alaninate was used in place of its antipode.
8. In this case, the intermediate tert-butyl (1-oxo-7-phenyl-1,2,4,5-tetrahydro[1,2,4]triazolo[4,3-a]quinolin-4-yl)carbamate was purified using silica gel chromatography (Eluents: 1:1 ethyl acetate in heptane followed by ethyl acetate); protecting group removal was then effected using 5 M hydrogen chloride in 2-propanol.
9. 1-Bromo-4-(2-methoxyethoxy)-2-nitrobenzene was prepared via Mitsunobu reaction of 4-bromo-3-nitrophenol with 2-methoxyethanol.
10. In this case, no chiral separation was carried out.
11. 1-Benzyl-5-bromo-2-methoxy-4-nitrobenzene (see M. M. Claffey et al., PCT Int. Appl. 2010, WO 2010146488 A1, Dec. 23, 2010) was reduced to 4-benzyl-2-bromo-5-methoxyaniline using tin(II) chloride. This compound was converted to tert-butyl [(3S)-6-benzyl-7-methoxy-2-thioxo-1,2,3,4-tetrahydroquinolin-3-yl]carbamate using the chemistry described in Example 1; in this case, methyl N-(tert-butoxycarbonyl)-3-iodo-L-alaninate was used in place of its antipode.
12. The reported NMR was acquired using the free base of the Example.
13. 2-Methyl-1-nitro-4-phenoxybenzene was brominated with N-bromosuccinimide to provide 2-(bromomethyl)-1-nitro-4-phenoxybenzene; reaction of this compound with tert-butyl N-(diphenylmethylidene)glycinate under the conditions described by E. J. Corey et al., J. Am. Chem. Soc. 1997, 119, 12414-12415 provided tert-butyl (2S)-2-(diphenylmethylidene)amino-3-(2-nitro-5-phenoxyphenyl)propanoate. Protecting group cleavage with concentrated hydrochloric acid afforded the requisite (2S)-2-amino-3-(2-nitro-5-phenoxyphenyl)propanoic acid.

### Example AA

### KAT II Inhibition Spectra Assay

Formation of kynurenic acid (KYNA) is indirectly assessed by a decrease in light absorbance at 370 nm (OD370) as the L-kynurenine (KYN) substrate is converted by the human KAT II (hKAT II) enzyme into KYNA. An inhibitor would therefore inhibit the decrease in OD370.

The protocol was performed by placing the following reagents into a Costar 384 well black plate (30 μL total assay volume/well):
10 μL of 3× concentrated compound;
10 μL of 3× concentrated substrate mix (BGG (Sigma G-5009); 3 mM L-Kynurenine in 150 mM Tris Acetate (Sigma K3750); 3 mM α-ketoglutaric acid in 150 mM Tris Acetate (Sigma K2010); and 210 μM pyridoxal 5-phosphate (PLP) in 150 mM Tris Acetate (Sigma 9255)); and
10 μL of 3× concentrated enzyme (15 nM enzyme in 150 mM Tris Acetate with 0.3% bovine serum).

Plates were sealed and incubated at 37° C. for 15-20 h before reading OD370 on a SpectraMax Plus plate reader. IC₅₀s were generated by comparing the efficacy of compounds across a concentration range to inhibit a reduction in the OD370 value relative to assay wells with DMSO added in place of concentrated compound. Biological data for the Examples may be found in Table 2.

TABLE 2

| | KATII IC₅₀ | |
|---|---|---|
| | (nM, single | |
| | determination | |
| Example | unless where | |
| Number | indicated) | IUPAC Name |
| 1 | 188^{a} | 4—amino—7—(3—methoxyphenoxy)—4,5— |
| | | dihydro[1,2,4]triazolo[4,3—a]quinolin—1 |
| | | (2H)—one ENT—1, hydrochloride salt |
| 2 | 59^{a} | 6—amino—3—phenoxy—6,8—dihydro[1,2,4]triazolo |
| | | [4,3—a][1,8]naphthyridin—9(5H)—one ENT—1 |
| 3 | 91.4 | 4—amino—7—[3—(trifluoromethyl)phenoxy]—4,5— |
| | | dihydro[1,2,4]triazolo[4,3—a]quinolin—1(2H)—one |
| | | hydrochloride salt |
| 4 | 107^{a} | 7—(3—methoxyphenoxy)—4,5-dihydro[1,2,4]triazolo |
| | | [4,3—a]quinolin—4—amine ENT—1 |
| 5 | 1240^{a} | 7—(3—methoxyphenoxy)—4,5-dihydro[1,2,4]triazolo |
| | | [4,3—a]quinolin—4—amine ENT—2 |
| 6 | 390 | (4S)—4—amino—7—(3—methoxyphenoxy)—4,5— |
| | | dihydro[1,2,4]oxadiazolo[4,3—a]quinolin—1—one, |
| | | hydrochloride salt |
| 7 | 413^{a} | 7—phenoxy—4,5—dihydro[1,2,4]triazolo[4,3— |
| | | a][1,7]naphthyridin—4—amine, hydrochloride salt |
| 8 | 286^{a} | 3—phenoxy—5,6—dihydro[1,2,4]triazolo[4,3— |
| | | a][1,8]naphthyridin—6—amine ENT—1 |
| 9 | 1310^{a} | 3—phenoxy—5,6—dihydro[1,2,4]triazolo[4,3— |
| | | a][1,8]naphthyridin—6—amine ENT—2 |
| 10 | 185 | 7—[3—(trifluoromethyl)phenoxy]—4,5— |
| | | dihydro[1,2,4]triazolo[4,3—a]quinolin—4—amine, |
| | | hydrochloride salt |
| 11 | 506 | 4—amino—7—(2-chlorophenyl)—4,5—dihydro[1,2,4] |
| | | triazolo[4,3—a]quinolin—1(2H)—one, |
| | | trifluoroacetate salt |
| 12 | 334 | 4—amino—7—(isoquinolin—5—yl)—4,5—dihydro[1,2,4] |
| | | triazolo[4,3—a]quinolin—1(2H)—one, |
| | | trifluoroacetate salt |
| 13 | 673 | 4—amino—7—(5—methoxypyriclin—3—yl)—4,5— |
| | | dihydro[1,2,4]triazolo[4,3—a]quinolin—1(2H)—one, |
| | | trifluoroacetate salt |
| 14 | 156 | 4—amino—7—(3—chlorophenyl)—4,5—dihydro[1,2,4] |
| | | triazolo[4,3—a]quinolin—1(2H)—one, |
| | | trifluoroacetate salt |
| 15 | 262 | 4—amino—7(1—methyl—1H—indazol—4—yl)—4,5— |
| | | dihydro[1,2,4]triazolo[4,3—a]quinolin—1(2H)—one, |
| | | trifluoroacetate salt |
| 16 | 664 | 4—amino—7—(quinolin—5—yl)—4,5—dihydro[1,2,4] |
| | | triazolo[4,3—a]quinolin—1(2H)—one, |
| | | trifluoroacetate salt |
| 17 | 1830 | 4—amino—7—(2—ethoxypyridin—4—yl)—4,5— |
| | | dihydro[1,2,4]triazolo[4,3—a]quinolin—1(2H)—one, |
| | | trifluoroacetate salt |
| 18 | 1360 | 4—amino—7—(1—methyl—1H—pyrazol—4—yl)—4,5— |
| | | dihydro[1,2,4]triazolo[4,3—a]quinolin—1(2H)—one, |
| | | trifluoroacetate salt |
| 19 | 751 | 4—amino—7—(2—methoxyphenyl)—4,5— |
| | | dihydro[1,2,4]triazolo[4,3—a]quinolin—1(2H)—one, |
| | | trifluoroacetate salt |
| 20 | 546 | 4—amino—7—(pyridin—3—yl)—4,5—dihydro[1,2,4] |
| | | triazolo[4,3—a]quinolin—1(2H)—one, |
| | | trifluoroacetate salt |
| 21 | 1250 | 4—amino—7—[2—chloro—5—(trifluoromethyl)phenyl]—4,5— |
| | | dihydro[1,2,4]triazolo[4,3—a]quinolin—1(2H)—one, |
| | | trifluoroacetate salt |
| 22 | 1900 | 4—amino—7—(6—methoxypyridin—3—yl)—4,5— |
| | | dihydro[1,2,4]triazolo[4,3—a]quinolin—1(2M—one, |
| | | trifluoroacetate salt |
| 23 | 684 | 4—amino—7—[3—(trifluoromethoxy)pheny1]—4,5— |
| | | dihydro[1,2,4]triazolo[4,3—a]quinolin—1(2H)—one, |
| | | trifluoroacetate salt |
| 24 | 1080 | 4—amino—7—(4—fluorophenyl)—4,5—dihydro[1,2,4] |
| | | triazolo[4,3—a]quinolin—1(2H)—one. |
| | | trifluoroacetate salt |
| 25 | 91.9 | 7—(3—acetylphenyl)—4—amino—4,5—dihydro[1,2,4] |
| | | triazolo[4,3—a]quinolin—1(2H)—one, |
| | | trifluoroacetate salt |
| 26 | 364 | 4—amino—7—(2,3-difluorophenyl)—4,5— |
| | | dihydro[1,2,4]triazolo[4,3—a]quinolin—1(2H)—one, |
| | | trifluoroacetate salt |
| 27 | 215 | 4—amino—7—[3—(trifluoromethyl)phenyl]—4,5— |
| | | dihydro[1,2,4]triazolo[4,3-a]quinolin—1(2H)—one, |
| | | trifluoroacetate salt |
| 28 | 357 | 4—amino—7—(3—methoxyphenyl)—4,5— |
| | | dihydro[1,2,4]triazolo[4,3—a]quinolin—1(2H)—one, |
| | | trifluoroacetate salt |
| 29 | 361 | 4—amino—7—(2—fluorophenyl)—4,5—dihydro[1,2,4] |
| | | triazolo[4,3—a]quinolin—1(2H)—one, |
| | | trifluoroacetate salt |
| 30 | 534 | 4—amino—7—[3—(propan—2—yloxy)phenyl]—4,5— |
| | | dihydro[1,2,4]triazolo[4,3—a]quinolin—1(2H)—one, |
| | | trifluoroacetate salt |
| 31 | 660 | 4—amino—7—(2—methylphenyl)—4,5—dihydro[1,2,4] |
| | | triazolo[4,3—a]quinolin—1(2H)—one, |
| | | trifluoroacetate salt |
| 32 | 302 | 4—amino—7—(5—fluoro—2—methoxyphenyl)—4,5— |
| | | dihydro[1,2,4]triazolo[4,3—a]quinolin—1(2H)—one, |
| | | trifluoroacetate salt |
| 33 | 197 | 4—amino—7—(2—methoxypyridin—3—yl)—4,5— |
| | | dihydro[1,2,4]triazolo[4,3—a]quinolin—1(2H)—one, |
| | | trifluoroacetate salt |
| 34 | 415 | 4—amino—7—(3—fluorophenyl)—4,5—dihydro[1,2,4] |
| | | triazolo[4,3—a]quinolin—1(2H)—one, |
| | | trifluoroacetate salt |
| 35 | 1,380 | 4—amino—7—(2,4—dichlorophenyl)—4,5— |
| | | dihydro[1,2,4]triazolo[4,3—a]quinolin—1(2H)—one, |
| | | trifluoroacetate salt |
| 36 | 632 | 4—amino—7—(1—methyl—1H—pyrazol—5—yl)—4,5— |
| | | dihydro[1,2,4]triazolo[4,3—a]quinolin—1(2H)—one, |
| | | trifluoroacetate salt |
| 37 | 527 | 4—amino—7—[2—(trifluoromethoxy)phenyl]—4,5— |
| | | dihydro[1,2,4]triazolo[4,3—a]quinolin—1(2H)—one, |
| | | trifluoroacetate salt |
| 38 | 591 | 4—amino—7—(2,5-dimethylphenyl)—4,5— |
| | | dihydro[1,2,4]triazolo[4,3—a]quinolin—1(2H)—one, |
| | | trifluoroacetate salt |
| 39 | 968 | 4—amino—7—(5—chloro—2—ethoxyphenyl)—4,5— |
| | | dihydro[1,2,4]triazolo[4,3—a]quinolin—1(2H)—one, |
| | | trifluoroacetate salt |
| 40 | 320 | 4—amino—7—[2—(trifluoromethyl)phenyl]—4,5— |
| | | dihydro[1,2,4]triazolo[4,3—a]quinolin—1(2H)—one, |
| | | trifluoroacetate salt |
| 41 | 1080 | 4—amino—7—(2—methyl—2H—indazol—4—yl)—4,5— |
| | | dihydro[1,2,4]triazolo[4,3—a]quinolin—1(2H)—one, |
| | | trifluoroacetate salt |
| 42 | 849 | 4—amino—7—(6—fluoropyridin—3—yl)—4,5— |
| | | dihydro[1,2,4]triazolo[4,3—a]quinolin—1(2H)—one, |
| | | trifluoroacetate salt |
| 43 | 263 | 4—amino—7—(6—fluoro—5—methylpyridin—3—yl)—4,5— |
| | | dihydro[1,2,4]triazolo[4,3—a]quinolin—1(2H)—one, |
| | | trifluoroacetate salt |
| 44 | 426^{a} | 4—amino—7—benzyl—4,5—dihydro[1,2,4]triazolo |
| | | [4,3—a]quinolin—1(2H)—one, |
| | | hydrochloride salt |
| 45 | 790^{a} | 4—amino—7—phenyl—4,5-dihydro[1,2,4]triazolo |
| | | [4,3—a]quinolin—1(2H)—one, |
| | | hydrochloride salt |
| 46 | 1130^{a} | 7—benzyl—8—methoxy—4,5—dihydro[1,2,4]triazolo |
| | | [4,3—a]quinolin—4—amine, hydrochloride salt |
| 47 | 637 | 4—amino—7—benzyl—8—methoxy—4,5—dihydro |
| | | [1,2,4]triazolo[4,3—a]quinolin—1(2H)—one, |
| | | hydrochloride salt |
| 48 | 83.7^{a} | 4—amino—7—phenoxy—4,5—dihydro[1,2,4]triazolo |
| | | [4,3—a]quinolin—1(2H)—one, hydrochloride salt |
| C9 | 3,720^{a} | 4—amino—7—(3—methoxyphenoxy)—4,5— |
| | | dihydro[1,2,4]triazolo[4,3—a]quinolin— |
| | | 1(2H)—one ENT—2, hydrochloride salt |

a. IC₅₀ value represents the geometric mean of 2-4 determinations.

1. A compound of Formula I: 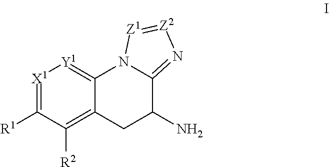
or a pharmaceutically acceptable salt thereof, wherein: [symbol]
represents a single bond or a double bond;
X¹ is CR³ or N;
Y¹ is CR⁴ or N;
Z¹ is CR⁵ or C(═O);
Z² is N, NH, or O;
R¹ is Q¹, —O-Q¹, or —CH₂-Q¹;
R² is H, OH, —CN, halogen, optionally substituted C₁₋₄ alkyl, or optionally substituted C₁₋₄ alkoxy;
each of R³ and R⁴ is independently H, OH, —CN, halogen, optionally substituted C₁₋₄ alkyl, or optionally substituted C₁₋₄ alkoxy;
R⁵ is H, OH, —CN, C₁₋₃ alkyl optionally substituted with one or more halogen, or C₁₋₃ alkoxy optionally substituted with one or more halogen; and
Q¹ is optionally substituted phenyl or optionally substituted 5- to 10-membered heteroaryl.

2. A compound of Formula Ia, Ib, or Ic: 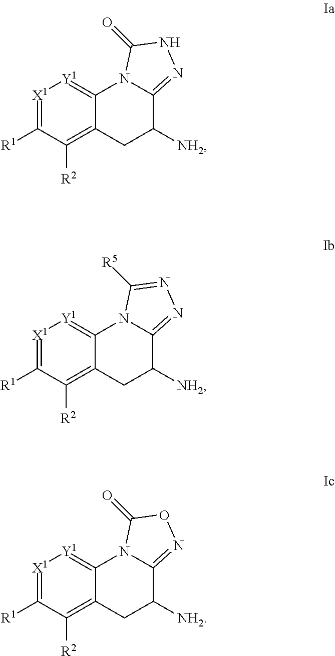
or a pharmaceutically acceptable salt thereof, wherein:
X¹ is CR³ or N;
Y¹ is CR⁴ or N;
R¹ is Q¹, —O-Q¹, or —CH₂-Q¹;
R² is H, OH, —CN, halogen, optionally substituted C₁₋₄ alkyl, or optionally substituted C₁₋₄ alkoxy;
each of R³ and R⁴ is independently H, OH, —CN, halogen, optionally substituted C₁₋₄ alkyl, or optionally substituted C₁₋₄ alkoxy;
R⁵ is H, OH, —CN, C₁₋₃ alkyl optionally substituted with one or more halogen, or C₁₋₃ alkoxy optionally substituted with one or more halogen; and
Q¹ is optionally substituted phenyl or optionally substituted 5- to 10-membered heteroaryl.

3. The compound according to claim 2, or a pharmaceutically acceptable salt thereof, wherein R⁵ is H, OH, methyl optionally substituted with one or more halogen, or methoxy optionally substituted with one or more halogen.

4. (canceled)

5. The compound according to claim 2, or a pharmaceutically acceptable salt thereof, wherein R⁵ is H or OH.

6. The compound according to claim 2, or a pharmaceutically acceptable salt thereof, wherein X¹ is CR³ and Y¹ is CR⁴; or wherein X¹ is CR³ and Y¹ is N; or wherein X¹ is N and Y¹ is CR⁴.

7-8. (canceled)

9. A compound of Formula Ia-1, Ia-2, Ib-1, Ib-2, Ib-3, or Ic-1: 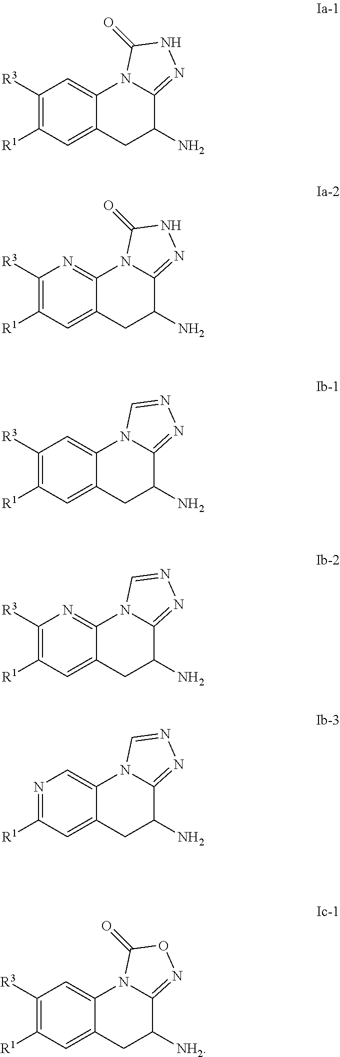
or a pharmaceutically acceptable salt thereof, wherein:
R¹ is Q¹, —O-Q¹, or —CH₂-Q¹;
R³ is H, OH, —CN, halogen, optionally substituted C₁₋₄ alkyl, or optionally substituted C₁₋₄ alkoxy; and
Q¹ is optionally substituted phenyl or optionally substituted 5- to 10-membered heteroaryl.

10. The compound according to claim 9, or a pharmaceutically acceptable salt thereof, wherein R³ is H.

11. The compound according to claim 9, or a pharmaceutically acceptable salt thereof, wherein:
R¹ is —O-Q¹; and
Q¹ is optionally substituted phenyl.

12. The compound according to claim 9, or a pharmaceutically acceptable salt thereof, wherein:
R¹ is —O-Q¹; and
Q¹ is phenyl optionally substituted with one or more substituents each independently selected from the group consisting of —CN, halogen, C₁₋₄ alkyl optionally substituted with one or more halogen, C₁₋₄ alkoxy optionally substituted with one or more halogen, and —C(═O)—(C₁₋₄ alkyl).

13. The compound according to claim 9, or a pharmaceutically acceptable salt thereof, wherein:
R¹ is —O-Q¹; and
Q¹ is phenyl optionally substituted with up to two substituents each independently selected from the group consisting of —CN, halogen, C₁₋₄ alkyl optionally substituted with one or more halogen, C₁₋₄ alkoxy optionally substituted with one or more halogen, and —C(═O)—(C₁₋₄ alkyl), and wherein each substituent on the phenyl is at one meta- or ortho-position.

14. The compound according to claim 9, or a pharmaceutically acceptable salt thereof, wherein:
R¹ is —O-Q¹; and
Q¹ is phenyl optionally substituted at one meta-position with —CN, halogen, C₁₋₄ alkyl optionally substituted with one or more halogen, C₁₋₄ alkoxy optionally substituted with one or more halogen, or —C(═O)—(C₁₋₄ alkyl).

15. The compound according to claim 9, or a pharmaceutically acceptable salt thereof, wherein R¹ is Q¹ or —CH₂-Q¹.

16. The compound according to claim 9, or a pharmaceutically acceptable salt thereof, wherein R¹ is optionally substituted phenyl or benzyl, wherein the phenyl moiety of the benzyl is optionally substituted phenyl.

17. The compound according to claim 9, or a pharmaceutically acceptable salt thereof, wherein R¹ is phenyl or benzyl, wherein the phenyl or the phenyl moiety of the benzyl is optionally substituted with one or more substituents each independently selected from the group consisting of —CN, halogen, C₁₋₄ alkyl optionally substituted with one or more halogen, C₁₋₄ alkoxy optionally substituted with one or more halogen, and —C(═O)—(C₁₋₄ alkyl).

18. The compound according to claim 9, or a pharmaceutically acceptable salt thereof, wherein R¹ is phenyl optionally substituted with up to two substituents each independently selected from the group consisting of —CN, halogen, C₁₋₄ alkyl optionally substituted with one or more halogen, C₁₋₄ alkoxy optionally substituted with one or more halogen, and —C(═O)—(C₁₋₄ alkyl).

19. (canceled)

20. The compound according to claim 9, or a pharmaceutically acceptable salt thereof, wherein R¹ is optionally substituted 5- to 10-membered heteroaryl.

21. The compound according to claim 9, or a pharmaceutically acceptable salt thereof, wherein R¹ is pyridinyl, pyrazolyl, indolyl, or indazolyl, each optionally substituted with one or more substituents each independently selected from the group consisting of halogen, C₁₋₄ alkyl optionally substituted with one or more halogen, C₁₋₄ alkoxy optionally substituted with one or more halogen, and —C(═O)—(C₁₋₄ alkyl).

22. The compound according to claim 1 wherein the compound is selected from the group consisting of:
4-amino-7-(3-methoxyphenoxy)-4,5-dihydro[1,2,4]triazolo[4,3-a]quinolin-1(2H)-one;
6-amino-3-phenoxy-6,8-dihydro[1,2,4]triazolo[4,3-a][1,8]naphthyridin-9(5H)-one;
4-amino-7-[3-(trifluoromethyl)phenoxy]-4,5-dihydro[1,2,4]triazolo[4,3-a]quinolin-1(2H)-one;
7-(3-methoxyphenoxy)-4,5-dihydro[1,2,4]triazolo[4,3-a]quinolin-4-amine;
7-[3-(trifluoromethyl)phenoxy]-4,5-dihydro[1,2,4]triazolo[4,3-a]quinolin-4-amine;
4-amino-7-(3-chlorophenyl)-4,5-dihydro[1,2,4]triazolo[4,3-a]quinolin-1(2H)-one;
7-(3-acetylphenyl)-4-amino-4,5-dihydro[1,2,4]triazolo[4,3-a]quinolin-1(2H)-one;
4-amino-7-[3-(trifluoromethyl)phenyl]-4,5-dihydro[1,2,4]triazolo[4,3-a]quinolin-1(2H)-one;
4-amino-7-(2-methoxypyridin-3-yl)-4,5-dihydro[1,2,4]triazolo[4,3-a]quinolin-1(2H)-one; and
4-amino-7-phenoxy-4,5-dihydro[1,2,4]triazolo[4,3-a]quinolin-1(2H)-one,
or a pharmaceutically acceptable salt thereof.

23. A pharmaceutical composition comprising a compound according to claim 1, or a pharmaceutically acceptable salt thereof, and a pharmaceutically acceptable carrier.

24. A method for treating a condition or disorder in a mammal wherein the method comprises administering to said mammal a therapeutically effective amount of a compound according to claim 1 or a pharmaceutically acceptable salt thereof, and wherein the condition or disorder is selected from the group consisting of acute neurological and psychiatric disorders; stroke; cerebral ischemia; spinal cord trauma; cognitive impairment; head trauma; perinatal hypoxia; cardiac arrest; hypoglycemic neuronal damage; dementia; Alzheimer's disease; Huntington's Chorea; amyotrophic lateral sclerosis; ocular damage; retinopathy; cognitive disorders; idiopathic and drug-induced Parkinson's disease; muscular spasms and disorders associated with muscular spasticity; epilepsy; convulsions; migraine; urinary incontinence; substance tolerance; substance withdrawal; psychosis; schizophrenia; negative symptoms associated with schizophrenia; autism; bipolar disorder; depression; cognitive impairment associated with depression; cognitive impairment associated with cancer therapy; anxiety; mood disorders; inflammatory disorders; sepsis; cirrhosis; cancer and/or tumors associated with immune response escape; trigeminal neuralgia; hearing loss; tinnitus; macular degeneration of the eye; emesis; brain edema; pain; tardive dyskinesia; sleep disorders; attention deficit/hyperactivity disorder; attention deficit disorder; disorders that comprise as a symptom a deficiency in attention and/or cognition; and conduct disorder.

25. (canceled)
